SEQUENCE LISTING (19 sequences)

SEQ ID NO: 1 - protein, 5 aa, synthetic construct
RFALH

SEQ ID NO: 2 - protein, 17 aa, synthetic construct
WIDPNTGTPTFAQGVTG

SEQ ID NO: 3 - protein, 18 aa, synthetic construct
SLGYCDSDICYPNWIFDN

SEQ ID NO: 4 - protein, 127 aa, synthetic construct
QVQLVQSGSELKKPGASVKVSCKASGYTFTRFALHWVRQAPGQGLEWMGWIDPNTGTPTFAQGVTGRFVFSLDTSVTTAYLQISSLKAEDTAVYYCARSLGYCDSDICYPNWIFDNWGQGTLVTVSS

SEQ ID NO: 5 - protein, 463 aa, synthetic construct
QVQLVQSGSELKKPGASVKVSCKASGYTFTRFALHWVRQAPGQGLEWMGWIDPNTGTPTFAQGVTGRFVFSLDTSVTTAYLQISSLKAEDTAVYYCARSLGYCDSDICYPNWIFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGSEQIDNO

SEQ ID NO: 6 - protein, 5 aa, synthetic construct
IYAMT

SEQ ID NO: 7 - protein, 17 aa, synthetic construct
VISGSGGTTYYADSVKG

SEQ ID NO: 8 - protein, 13 aa, synthetic construct
RGQYRDIVGATDY

SEQ ID NO: 9 - protein, 122 aa, synthetic construct
EVQLVESGGGLVQPGGSLRLSCAASGFTFDIYAMTWVRQAPGKGLEWVSVISGSGGTTYYADSVKGRFTISRDNSKNTLYLQMNSLRAEDTAVYYCARRGQYRDIVGATDYWGQGTLVTVSS

SEQ ID NO: 10 - protein, 458 aa, synthetic construct
EVQLVESGGGLVQPGGSLRLSCAASGFTFDIYAMTWVRQAPGKGLEWVSVISGSGGTTYYADSVKGRFTISRDNSKNTLYLQMNSLRAEDTAVYYCARRGQYRDIVGATDYWGQGTLVTVSSASTKGPSVFPLAPSSKSTSGGTAALGCLVKDYFPEPVTVSWKSGALTSGVHTFPAVLQSSGLYSLSSVVTVPSSSLGTQTYICKVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGSEQIDNO

SEQ ID NO: 11 - protein, 6 aa, synthetic construct
QSISSY

SEQ ID NO: 12 - skipped

SEQ ID NO: 13 - protein, 9 aa, synthetic construct
QQSYSTPPT

SEQ ID NO: 14 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 15 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 451 aa, synthetic construct
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 214 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 461 aa, synthetic construct
EVQLVESGGGVVQPGRSLRLSCAASGFTFSNYGMHWVRQAPGKGLEWVAVISYDGSTKYSADSLKGRFTISRDNSKNTLYLQMNSLRADDTAVYYCAKEGWSFDSSGYRSWFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSEQIDNO

SEQ ID NO: 19 - protein, 214 aa, synthetic construct; identical to SEQ ID NO: 15

(19) United States
(12) Patent Application Publication
(10) Pub. No.: US 2024/0343805 A1
(43) Pub. Date: Oct. 17, 2024

(54) USE OF AN ANTI-TGFßR2/PD-1 BISPECIFIC ANTIBODY

(71) Applicant: Incyte Corporation, Wilmington, DE (US)

(72) Inventors: Liang-Chuan Wang, Wayne, PA (US); Chifei Sun, King of Prussia, PA (US); Ekaterine Asatiani, Founex (CH)

(21) Appl. No.: 18/633,748

(22) Filed: Apr. 12, 2024

Related U.S. Application Data
(60) Provisional application No. 63/496,205, filed on Apr. 14, 2023.

(51) Int. Cl. C07K 16/28; A61K 9/00; A61K 39/00; A61P 35/00

(52) U.S. Cl. CPC C07K 16/2818; A61K 9/0019; A61P 35/00; C07K 16/2863; A61K 2039/505; A61K 2039/545; C07K 2317/31; C07K 2317/565

(57) ABSTRACT

The present disclosure describes a bispecific antibody targeting human programmed death-1 (PD-1) and human transforming growth factor β receptor 2 (TGFβR2) for use in the treatment of disorder such as non-small cell lung cancer (NSCLC), squamous cell carcinoma of the head and neck (SCCHN), cervical squamous cell carcinoma and endocervical adenocarcinoma (CESC), ovarian cancer, breast cancer, bladder cancer, renal cell carcinoma, melanoma, gastric adenocarcinoma, esophageal cancer, gastroesophageal adenocarcinoma, malignant pleural mesothelioma, pancreatic adenocarcinoma, and colorectal cancer (CRC).

### CLAIM OF PRIORITY

This application claims the benefit of U.S. Provisional Patent Application No. 63/496,205, filed on Apr. 14, 2023, which is incorporated by reference herein in its entirety.

### SEQUENCE LISTING

This application contains a Sequence Listing that has been submitted electronically as an XML file named 20443-0795001.XML. The XML file, created on Apr. 4, 2024, is 19,545 bytes in size. The material in the XML file is hereby incorporated by reference in its entirety.

### TECHNICAL FIELD

The present invention is directed to treating cancer by administering to a subject a bispecific antibody that binds to human TGFβR2 and human PD-1.

### BACKGROUND

Cancer is among the leading causes of death worldwide. Many patients are diagnosed with advanced disease, have no response to treatment, or have a response to treatment that is followed by disease progression. Thus, there is a need for therapies targeting cancer.

### SUMMARY

The present disclosure is based, at least in part, on the development of cancer treatments using a bispecific antibody that that binds to human TGFβR2 and human PD-1.

Accordingly, aspects of the present disclosure provide a method of treating a disorder in a human subject in need thereof, wherein the disorder is selected from the group consisting of a non-small cell lung cancer (NSCLC), a squamous cell carcinoma of the head and neck (SCCHN), a cervical squamous cell carcinoma and endocervical adenocarcinoma (CESC), an ovarian cancer, a breast cancer, a bladder cancer, a renal cell carcinoma, a melanoma, a gastric adenocarcinoma, an esophageal cancer, a gastroesophageal adenocarcinoma, a malignant pleural mesothelioma, a pancreatic adenocarcinoma, and a colorectal cancer (CRC), wherein the method comprises administering to the human subject a therapeutically effective amount of a bispecific antibody that binds to human programmed death-1 (PD-1) and human transforming growth factor β receptor 2 (TGFβR2), wherein the bispecific antibody comprises:
an anti-human PD-1 binding domain comprising a PD-1 heavy chain variable region and a PD-1 light chain variable region, wherein the PD-1 heavy chain variable region comprises heavy chain CDR1 (HCDR1) comprising the amino acid sequence RFALH (SEQ ID NO:1), heavy chain CDR2 (HCDR2) comprising the amino acid sequence WIDPNTGTPTFAQGVTG (SEQ ID NO:2), and heavy chain CDR3 (HCDR3) comprising the amino acid sequence SLGYCDSDICYPNWIFDN (SEQ ID NO:3), and wherein the PD-1 light chain variable region comprises light chain CDR1 (LCDR1) comprising the amino acid sequence QSISSY (SEQ ID NO:11), light chain CDR2 (LCDR2) comprising the amino acid sequence AAS (SEQ ID NO:12), and light chain CDR3 (LCDR3) comprising the amino acid sequence QQSYSTPPT (SEQ ID NO:13); and
an anti-human TGFβR2 binding domain comprising a TGFβR2 heavy chain variable region and a TGFβR2 light chain variable region, wherein the TGFβR2 heavy chain variable region comprises HCDR1 comprising the amino acid sequence IYAMT (SEQ ID NO:6), HCDR2 comprising the amino acid sequence VISGSGGTTYYADSVKG (SEQ ID NO:7), and HCDR3 comprising the amino acid sequence RGQYRDIVGATDY (SEQ ID NO:8), and wherein the TGFβR2 light chain variable region comprises LCDR1 comprising the amino acid sequence QSISSY (SEQ ID NO:11), LCDR2 comprising the amino acid sequence AAS (SEQ ID NO:12), and LCDR3 comprising the amino acid sequence QQSYSTPPT (SEQ ID NO:13).

In some embodiments, the PD-1 heavy chain variable region comprises the amino acid sequence QVQLVQSGSELKKPGASVKVSCKASGYTFTRFALHWVRQAPGQGLEWMGWIDPNT GTPTFAQGVTGRFVFSLDTSVTTAYLQISSLKAEDTAVYYCARSLGYCDSDICYPNWI FDNWGQGTLVTVSS (SEQ ID NO:4) and the TGFβR2 heavy chain variable region comprises the amino acid sequence EVQLVESGGGLVQPGGSLRLSCAASGFTFDIYAMTWVRQAPGKGLEWVSVISGSGG TTYYADSVKGRFTISRDNSKNTLYLQMNSLRAEDTAVYYCARRGQYRDIVGATDY WGQGTLVTVSS (SEQ ID NO:9).

In some embodiments, the PD-1 light chain variable region and the TGFβR2 light chain variable region each comprise the amino acid sequence DIQMTQSPSSLSASVGDRVTITCRASQSISSYLNWYQQKPGKAPKLLIYAASSLQSGV PSRFSGSGSGTDFTLTISSLQPEDFATYYCQQSYSTPPTFGQGTKVEIK (SEQ ID NO:14).

In some embodiments, the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO:15, the TGFβR2 heavy chain comprises the amino acid sequence set forth in SEQ ID NO: 10, and the TGFβR2 light chain light chain comprises the amino acid sequence set forth in SEQ ID NO:15.

In some embodiments, the human subject has a NSCLC. In some embodiments, the NSCLC has squamous or nonsquamous histology. In some embodiments, the NSCLC is advanced or metastatic.

In some embodiments, the human subject has a SCCHN. In some embodiments, the SCCHN comprises a primary squamous tumor of the oral cavity, oropharynx, hypopharynx, or larynx. In some embodiments, the SCCHN is advanced or metastatic.

In some embodiments, the human subject has a CESC. In some embodiments, the CESC is advanced or metastatic.

In some embodiments, the human subject has human papilloma virus (HPV)-positive SCCHN or HPV-positive CESC.

In some embodiments, the human subject has an ovarian cancer. In some embodiments, the ovarian cancer comprises ovarian epithelial carcinoma, fallopian tube carcinoma, primary peritoneal carcinoma, or carcinosarcoma. In some embodiments, the ovarian cancer is advanced or metastatic.

In some embodiments, the human subject has a breast cancer. In some embodiments, the breast cancer comprises a triple negative breast cancer. In some embodiments, the triple negative breast cancer comprises HER2-negative, ER-negative, and PgR-negative breast cancer. In some embodiments, the breast cancer is advanced or metastatic.

In some embodiments, the human subject has a bladder cancer. In some embodiments, the bladder cancer comprises urothelial carcinoma. In some embodiments, the bladder cancer is advanced or metastatic.

In some embodiments, the human subject has a renal cell carcinoma. In some embodiments, the renal cell carcinoma is advanced or metastatic.

In some embodiments, the human subject has a melanoma. In some embodiments, the melanoma is cutaneous malignant melanoma. In some embodiments, the melanoma is advanced or metastatic.

In some embodiments, the human subject has a gastric adenocarcinoma. In some embodiments, the gastric adenocarcinoma is advanced or metastatic.

In some embodiments, the human subject has an esophageal cancer. In some embodiments, the esophageal cancer is advanced or metastatic.

In some embodiments, the human subject has a gastroesophageal adenocarcinoma. In some embodiments, the gastroesophageal adenocarcinoma is advanced or metastatic.

In some embodiments, the human subject has a malignant pleural mesothelioma. In some embodiments, the malignant pleural mesothelioma is advanced or metastatic.

In some embodiments, the human subject has a pancreatic adenocarcinoma. In some embodiments, the pancreatic adenocarcinoma is advanced or metastatic.

In some embodiments, the human subject has a CRC. In some embodiments, the CRC comprises microsatellite stable colorectal cancer (MSS-CRC). In some embodiments, the CRC comprises deficient mismatch repair (dMMR)/high microsatellite instability (MSI-H) colorectal cancer (dMMR/MSI-H CRC). In some embodiments, the CRC is advanced or metastatic.

In some embodiments, the human subject has experienced disease progression after prior treatment. In some embodiments, the prior treatment comprises anti-PD-(L)1 therapy and/or anti-CTLA4 therapy.

In some embodiments, the disorder is nonamenable to curative treatments or procedures.

In some embodiments, the bispecific antibody is administered intravenously.

In some embodiments, the bispecific antibody is administered at a dose of about 100 mg, about 300 mg, about 900 mg, about 1500 mg, or about 2000 mg.

In some embodiments, the bispecific antibody is administered intravenously at a dose of about 100 mg, about 300 mg, about 900 mg, about 1500 mg, or about 2000 mg.

In some embodiments, the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO:15, the TGFβR2 heavy chain comprises the amino acid sequence set forth in SEQ ID NO:10, and the TGFβR2 light chain light chain comprises the amino acid sequence set forth in SEQ ID NO:15, and wherein the bispecific antibody is administered intravenously at a dose of about 100 mg, about 300 mg, about 900 mg, about 1500 mg, or about 2000 mg.

In some embodiments, the bispecific antibody is administered once every two weeks. In some embodiments, the bispecific antibody is administered intravenously once every two weeks at a dose of about 100 mg, about 300 mg, about 900 mg, about 1500 mg, or about 2000 mg. In some embodiments, the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO:15, the TGFβR2 heavy chain comprises the amino acid sequence set forth in SEQ ID NO:10, and the TGFβR2 light chain light chain comprises the amino acid sequence set forth in SEQ ID NO:15, and wherein the bispecific antibody is administered intravenously once every two weeks at a dose of about 100 mg, about 300 mg, about 900 mg, about 1500 mg, or about 2000 mg.

In some embodiments, the bispecific antibody is administered once every four weeks. In some embodiments, the bispecific antibody is administered intravenously once every four weeks at a dose of about 100 mg, about 300 mg, about 900 mg, about 1500 mg, or about 2000 mg. In some embodiments, the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO:15, the TGFβR2 heavy chain comprises the amino acid sequence set forth in SEQ ID NO:10, and the TGFβR2 light chain light chain comprises the amino acid sequence set forth in SEQ ID NO:15, and wherein the bispecific antibody is administered intravenously once every four weeks at a dose of about 100 mg, about 300 mg, about 900 mg, about 1500 mg, or about 2000 mg.

### BRIEF DESCRIPTION OF THE DRAWINGS

FIGS. 1A-IF are graphs of results obtained from in vitro cytotoxicity assays performed using co-cultures of renal cancer cells and T cells, which were cultured in the absence (FIGS. 1C-1D) or the presence of exogenous TGF-β (FIGS. 1A-1B: 0.1 ng/mL; FIGS. 1E-1F: 1.0 ng/mL).

FIGS. 2A-2B are graphs of tumor volume (FIG. 2A) and body weight (FIG. 2B) of mice subcutaneously inoculated with A375 human melanoma cells and then treated with various antibodies. Humanized mice bearing subcutaneous A375 human melanoma tumors were administered antibodies every 5 days beginning on Day 8, and tumors were measured twice weekly for tumor volume. Body weight was measured twice weekly. Data are displayed as mean±SEM.

FIGS. 3A-3H are graphs of tumor volume plotted against stem cell donor for humanized mice bearing A375 human melanoma tumors. Mice were untreated (FIG. 3A) or treated with IgG1 control antibody (FIG. 3B), atezolizumab (FIG. 3C), pembrolizumab (FIG. 3D), TGF1 antibody (FIG. 3E), pembrolizumab+TGF1 antibody (FIG. 3F), ANTIBODY A (FIG. 3G), or NIS793 (FIG. 3H). Donors are identified by numbers (0678, 0717, 0731, and 0687). The dotted line at 750 mm³ is an arbitrary line for visual aid.

FIG. 4 is a graph of tumor volume in mice subcutaneously inoculated with MDA-MB-231 human triple negative breast cancer (TNBC) cells and then treated with various antibodies.

FIGS. 5A-5B are graphs of tumor volume in mice subcutaneously inoculated with MC38 colorectal cancer cells and then treated with various antibodies (FIG. 5A). Cured mice were rechallenged with MC38 colorectal cancer cells and tumor volumes were monitored over time (FIG. 5B).

FIGS. 6A-6B are graphs from a flow cytometric analysis of CD8 T cells (FIG. 6A) and Tregs (FIG. 6B) in MC38 colorectal tumors harvested from transgenic hPD-1/hTGFβR2 mice after treatment with various antibodies.

FIGS. 7A-7C are representative photomicrographs (FIG. 7A) and graphs showing the CD8 cell density (FIG. 7B) and percent CD8 positive cells (FIG. 7C) from IHC analysis of MC38 colorectal tumors harvested from transgenic hPD-1/hTGFβR2 mice after treatment with ANTIBODY A (1 mg/kg or 10 mg/kg) or the control antibody RSV.

FIG. 8 is a graph of IFNγ-producing Ly95 T cells in co-cultures with A549-eso cells in the presence of tumor digests from NSCLC patients. Co-cultures were treated with the indicated antibodies.

FIGS. 9A-9B are graphs of IFNγ concentration (pg/mL) in two different ovarian ascites samples treated with various antibodies for 4 days. FIG. 9A: ovarian ascites sample 1; FIG. 9B: ovarian ascites sample 2.

### DETAILED DESCRIPTION

The present disclosure provides, in part, methods of treating cancer by administering a bispecific antibody targeting TGFβR2 and PD-1. While not wishing to be bound by theory, it is believed that concurrent targeting of TGFβR2 and PD-1 can alleviate immunosuppressive pathways to promote cytotoxic T-lymphocyte function and T-cell memory for effective cancer elimination while minimizing toxicity associated with systemic TGFβR2 blockage.

### Anti-TGFβR2/PD-1 Bispecific Antibodies

Transforming growth factor β is a multifunctional cytokine that acts in a cell- and context-dependent manner as a tumor promoter or tumor suppressor. Within healthy cells, TGFβ halts the cell cycle at the growth 1 phase, resulting in reduced proliferation and induction of differentiation while it may also promote apoptosis. In cancer cells, the TGFβ signaling pathway is deregulated or altered, and TGFβ no longer has the ability to control cellular proliferation. In mammals, there are 3 highly homologous TGFβ isoforms: TGFβ1, TGFβ2, and TGFβ3. The most prevalent, TGFβ1, is expressed in the majority of human cancer types. In addition, TGFβ1 expression has been the isoform most closely correlated with TGFβ signaling activation compared with isoforms TGFβ2 and TGFβ3.

TGFβ is synthesized as an inactive precursor that must be activated to allow for engagement of a tetrameric receptor complex composed of TGFβR1 and TGFβR2. Transforming growth factor β receptor 2 is a membrane-bound serine/threonine kinase that binds TGFβ1 and TGFβ3 with relative high affinity and TGFβ2 with lower affinity. Formation of a heterodimeric complex with TGFβR1 is required for signaling transduction following binding of the TGFβ ligand. Activated TGFβR2 phosphorylates multiple serine and threonine residues in the intracellular domain of TGFβR1, leading to its activation. Activated TGFβR1 then mediates activation of a downstream signaling pathway involving SMAD proteins that regulate target gene expression.

Programmed Cell Death 1 protein (PD-1, also known as CD279) is a cell surface receptor expressed on CD4+ and CD8+ T cells, B cells, NK cells, and myeloid-derived cells. PD-1 binds to two distinct ligands, PD-L1 and PD-L2, which differ in their expression patterns. PD-L1 (also known as B7-H1 or CD274) is expressed on hematopoietic cells such as T cells, B cells, dendritic cells, and macrophages, as well as an array of peripheral tissues, and PD-L1 expression levels are inducible by interferons. In contrast, PD-L2 (also known as B7-DC or CD273) expression is generally restricted to professional APCs and inducible by IL-4 and IL-10, depending on the lineage subset of the APC.

Binding of PD-1 to either PD-L1 or PD-L2 on T cells or B cells results in clustering with TCRs or BCRs and transient association with SH2 domain-containing tyrosine phosphatase 2. In turn, this induces a negative signal by dephosphorylating effector molecules that drive positive TCR and BCR signaling. This includes CD28-mediated activation of PI3K and subsequently Akt, glucose metabolism, and the survival protein Bcl-XL. Overall, this results in the suppression of T-cell or B-cell activation, proliferation, and cytokine secretion. PD-1 expression on T cells following chronic viral infection and on tumor-infiltrating lymphocytes has been shown to result in immune dysfunction characteristic of exhaustion, while blockade of PD-1 signaling has been shown to enhance T-cell proliferation and restore immune responses.

ANTIBODY A is a human Fc-silenced IgG1 bispecific antibody that can simultaneously bind to both TGFβR2 and PD-1. ANTIBODY A is designed to block the PD-1 axis and selectively targets TGFβ signaling blockade on activated PD-1-expressing T cells.

The amino acid sequences of the ANTIBODY A heavy and light chains are shown below. ANTIBODY A contains two different heavy chains, a TGFβR2 heavy chain (which binds TGFβR2) and a PD-1 heavy chain (which binds to PD-1), and a common light chain that pairs with each of the TGFβR2 and PD-1 heavy chains. Complementarity-determining regions (CDRs) 1, 2, and 3 (HCDRs specified according to Kabat (see Kabat et al., Sequences of Proteins of Immunological Interest, U.S. Dept. of Health and Human Services, Public Health Service, National Institutes of Health, 1991, (OCoLC)1138727707) and LCDRs specified according to IMGT (see Giudicelli et al., IMGT/V-QUEST: IMGT standardized analysis of the immunoglobulin (IG) and T cell receptor (TR) nucleotide sequences. Cold Spring Harb Protoc 2011(6): 695-715)) of the variable heavy (VH) domain and the variable light (VL) domain are shown below. An antibody consisting of the TGFβR2 heavy chain amino acid sequence set forth in SEQ ID NO:10, the PD-1 heavy chain amino acid sequence set forth in SEQ ID NO:5, and the common light chain amino acid sequence set forth in SEQ ID NO:15 (one light chain paired with each of the heavy chains) is termed “ANTIBODY A.”

The heavy chain (HC) of the anti-PD-1 binding domain and anti-TGFβR2 binding domain of ANTIBODY A, as well as the common light chain amino acid sequence, are depicted in Table 1.

TABLE 1
ANTIBODY A Sequences

| Antibody | |
|---|---|
| Feature | Sequences |
| anti-PD- | QVQLVQSGSELKKPGASVKVSCKASGYTFTRFALHW |
| 1 HC | VRQAPGQGLEWMGWIDPNTGTPTFAQGVTGRFVFSL |
| | DTSVTTAYLQISSLKAEDTAVYYCARSLGYCDSDIC |
| | YPNWIFDNWGQGTLVTVSSASTKGPSVFPLAPSSKS |
| | TSGGTAALGCLVKDYFPEPVTVSWNSGALTSGVHTF |
| | PAVLQSSGLYSLSSVVTVPSSSLGTQTYICNVNHKP |
| | SNTKVDKRVEPKSCDKTHTCPPCPAPELGRGPSVFL |
| | FPPKPKDTLMISRTPEVTCVVVDVSHEDPEVKFNWY |
| | VDGVEVHNAKTKPREEQYNSTYRVVSVLTVLHQDWL |
| | NGKEYKCKVSNKALPAPIEKTISKAKGQPREPQVYT |
| | DPPSREEMTKNQVSLTCEVKGFYPSDIAVEWESNGQ |
| | PENNYKTTPPVLDSDGSFFLYSKLTVDKSRWQQGNV |
| | FSCSVMHEALHNHYTQKSLSLSPG |
| | (SEQ ID NO: 5) |
| anti- | EVQLVESGGGLVQPGGSLRLSCAASGFTFDIYAMTW |
| TGFβR2 | VRQAPGKGLEWVSVISGSGGTTYYADSVKGRFTISR |
| HC | DNSKNTLYLQMNSLRAEDTAVYYCARRGQYRDIVGA |
| | TDYWGQGTLVTVSSASTKGPSVFPLAPSSKSTSGGT |
| | AALGCLVKDYFPEPVTVSWKSGALTSGVHTFPAVLQ |
| | SSGLYSLSSVVTVPSSSLGTQTYICKVNHKPSNTKV |
| | DKRVEPKSCDKTHTCPPCPAPELGRGPSVFLFPPKP |
| | KDTLMISRTPEVTCVVVDVSHEDPEVKFNWYVDGVE |
| | VHNAKTKPREEQYNSTYRVVSVLTVLHQDWLNGKEY |
| | KCKVSNKALPAPIEKTISKAKGQPREPQVYTKPPSR |
| | EEMTKNQVSLKCLVKGFYPSDIAVEWESNGQPENNY |
| | KTTPPVLDSDGSFFLYSKLTVDKSRWQQGNVFSCSV |
| | MHEALHNHYTQKSLSLSPG (SEQ ID NO: 10) |
| common | DIQMTQSPSSLSASVGDRVTITCRASQSISSYLNWY |
| LC | QQKPGKAPKLLIYAASSLQSGVPSRFSGSGSGTDFT |
| | LTISSLQPEDFATYYCQQSYSTPPTFGQGTKVEIKR |
| | TVAAPSVEIFPPSDEQLKSGTASVVCLLNNFYPREA |
| | KVQWKVDNALQSGNSQESVTEQDSKDSTYSLSSTLT |
| | LSKADYEKHKVYACEVTHQGLSSPVTKSFNRGEC |
| | (SEQ ID NO: 15) |

The VH and VL, as well as the CDRs of each of the VH and VL, are depicted in Tables 2-3 for the anti-PD-1 binding domain and the anti-TGFβR2 binding domain of ANTIBODY A, respectively.

TABLE 2
Amino Acid Sequences of Anti-PD-1 Binding
Domain of ANTIBODY A

| Antibody | |
|---|---|
| Feature | Sequences |
| anti- | REALH (SEQ ID NO: 1) |
| PD-1 HC | |
| CDR1 | |
| anti- | WIDPNTGTPTFAQGVTG (SEQ ID NO: 2) |
| PD-1 HC | |
| CDR2 | |
| anti- | SLGYCDSDICYPNWIFDN (SEQ ID NO: 3) |
| PD-1 HC | |
| CDR3 | |
| common | QSISSY (SEQ ID NO: 11) |
| LC | |
| CDR1 | |
| common | AAS (SEQ ID NO: 12) |
| LC | |
| CDR2 | |
| common | QQSYSTPPT (SEQ ID NO: 13) |
| LC | |
| CDR3 | |
| anti- | QVQLVQSGSELKKPGASVKVSCKASGYTFTRFALHW |
| PD-1 VH | VRQAPGQGLEWMGWIDPNTGTPTFAQGVTGRFVFSL |
| | DTSVTTAYLQISSLKAEDTAVYYCARSLGYCDSDIC |
| | YPNWIFDNWGQGTLVTVSS (SEQ ID NO: 4) |
| common | DIQMTQSPSSLSASVGDRVTITCRASQSISSYLNWY |
| VL | QQKPGKAPKLLIYAASSLQSGVPSRFSGSGSGTDFT |
| | LTISSLQPEDFATYYCQQSYSTPPTFGQGTKVEIK |
| | (SEQ ID NO: 14) |

TABLE 3
Amino Acid Sequences of Anti-TGFβR2
Binding Domain of ANTIBODY A

| Antibody | |
|---|---|
| Feature | Sequences |
| anti- | IYAMT (SEQ ID NO: 6) |
| TGFβR2 | |
| HC CDR1 | |
| anti- | VISGSGGTTYYADSVKG (SEQ ID NO: 7) |
| TGFβR2 | |
| HC CDR2 | |
| anti- | RGQYRDIVGATDY (SEQ ID NO: 8) |
| TGFβR2 | |
| HC CDR3 | |
| common | QSISSY (SEQ ID NO: 11) |
| LC | |
| CDR1 | |
| common | AAS (SEQ ID NO:12) |
| LC | |
| CDR2 | |
| common | QQSYSTPPT (SEQ ID NO: 13) |
| LC | |
| CDR3 | |
| anti- | EVQLVESGGGLVQPGGSLRLSCAASGFTFDIYAMTW |
| TGFβR2 | VRQAPGKGLEWVSVISGSGGTTYYADSVKGRFTISR |
| VH | DNSKNTLYLQMNSLRAEDTAVYYCARRGQYRDIVGA |
| | TDYWGQGTLVTVSS (SEQ ID NO: 9) |
| common | DIQMTQSPSSLSASVGDRVTITCRASQSISSYLNWY |
| VL | QQKPGKAPKLLIYAASSLQSGVPSRFSGSGSGTDFT |
| | LTISSLQPEDFATYYCQQSYSTPPTFGQGTKVEIK |
| | (SEQ ID NO: 14) |

In some embodiments, the anti-human PD-1 binding domain of the bispecific antibody of the present disclosure comprises a heavy chain variable region comprising an HCDR1 comprising the amino acid sequence set forth in SEQ ID NO:1, an HCDR2 comprising the amino acid sequence set forth in SEQ ID NO:2, and an HCDR3 comprising the amino acid sequence set forth in SEQ ID NO:3. In some embodiments, the anti-human PD-1 binding domain of the bispecific antibody of the present disclosure comprises a heavy chain variable region comprising the amino acid sequence as set forth in SEQ ID NO:4, or having at least 80%, 85%, 90%, or 95% sequence identity thereto. In some embodiments, the anti-human PD-1 binding domain of the bispecific antibody of the present disclosure comprises a heavy chain variable region (1) comprising an HCDR1 comprising the amino acid sequence set forth in SEQ ID NO:1, an HCDR2 comprising the amino acid sequence set forth in SEQ ID NO:2, and an HCDR3 comprising the amino acid sequence set forth in SEQ ID NO:3, and (2) comprising the amino acid sequence as set forth in SEQ ID NO:4, or having at least 80%, 85%, 90%, or 95% sequence identity thereto.

In some embodiments, the anti-human TGFβR2 binding domain of the bispecific antibody of the present disclosure comprises a heavy chain variable region comprising an HCDR1 comprising the amino acid sequence set forth in SEQ ID NO:6, an HCDR2 comprising the amino acid sequence set forth in SEQ ID NO:7, and an HCDR3 comprising the amino acid sequence set forth in SEQ ID NO:8. In some embodiments, the anti-human TGFβR2 binding domain of the bispecific antibody of the present disclosure comprises a heavy chain variable region comprising the amino acid sequence as set forth in SEQ ID NO:9, or having at least 80%, 85%, 90%, or 95% sequence identity thereto. In some embodiments, the anti-human TGFβR2 binding domain of the bispecific antibody of the present disclosure comprises a heavy chain variable region (1) comprising an HCDR1 comprising the amino acid sequence set forth in SEQ ID NO:6, an HCDR2 comprising the amino acid sequence set forth in SEQ ID NO:7, and an HCDR3 comprising the amino acid sequence set forth in SEQ ID NO:8, and (2) comprising the amino acid sequence as set forth in SEQ ID NO:9, or having at least 80%, 85%, 90%, or 95% sequence identity thereto.

In some embodiments, the anti-human PD-1 binding domain of the bispecific antibody of the present disclosure comprises a heavy chain variable region comprising an HCDR1 comprising the amino acid sequence set forth in SEQ ID NO:1, an HCDR2 comprising the amino acid sequence set forth in SEQ ID NO:2, and an HCDR3 comprising the amino acid sequence set forth in SEQ ID NO:3, and the anti-human TGFβR2 binding domain of the bispecific antibody comprises a heavy chain variable region comprising an HCDR1 comprising the amino acid sequence set forth in SEQ ID NO:6, an HCDR2 comprising the amino acid sequence set forth in SEQ ID NO:7, and an HCDR3 comprising the amino acid sequence set forth in SEQ ID NO:8. In some embodiments, the anti-human PD-1 binding domain of the bispecific antibody of the present disclosure comprises a heavy chain variable region comprising the amino acid sequence as set forth in SEQ ID NO:4, or having at least 80%, 85%, 90%, or 95% sequence identity thereto, and the anti-human TGFβR2 binding domain of the bispecific antibody comprises a heavy chain variable region comprising the amino acid sequence as set forth in SEQ ID NO:9, or having at least 80%, 85%, 90%, or 95% sequence identity thereto. In some embodiments, the anti-human PD-1 binding domain of the bispecific antibody of the present disclosure comprises a heavy chain variable region (1) comprising an HCDR1 comprising the amino acid sequence set forth in SEQ ID NO:1, an HCDR2 comprising the amino acid sequence set forth in SEQ ID NO:2, and an HCDR3 comprising the amino acid sequence set forth in SEQ ID NO:3, and (2) comprising the amino acid sequence as set forth in SEQ ID NO:4, or having at least 80%, 85%, 90%, or 95% sequence identity thereto, and the anti-human TGFβR2 binding domain of the bispecific antibody comprises a heavy chain variable region (1) comprising an HCDR1 comprising the amino acid sequence set forth in SEQ ID NO:6, an HCDR2 comprising the amino acid sequence set forth in SEQ ID NO:7, and an HCDR3 comprising the amino acid sequence set forth in SEQ ID NO:8, and (2) comprising the amino acid sequence as set forth in SEQ ID NO:9, or having at least 80%, 85%, 90%, or 95% sequence identity thereto.

In some embodiments, the anti-human PD-1 binding domain of the bispecific antibody of the present disclosure comprises (1) a heavy chain variable region comprising an HCDR1 comprising the amino acid sequence set forth in SEQ ID NO:1, an HCDR2 comprising the amino acid sequence set forth in SEQ ID NO:2, and an HCDR3 comprising the amino acid sequence set forth in SEQ ID NO:3, and (2) a light chain variable region comprising an LCDR1 comprising the amino acid sequence set forth in SEQ ID NO:11, an LCDR2 comprising the amino acid sequence set forth in SEQ ID NO:12, and an LCDR3 comprising the amino acid sequence set forth in SEQ ID NO:13. In some embodiments, the anti-human PD-1 binding domain of the bispecific antibody of the present disclosure comprises (1) a heavy chain variable region comprising the amino acid sequence as set forth in SEQ ID NO:4, or having at least 80%, 85%, 90%, or 95% sequence identity thereto, and (2) a light chain variable region comprising the amino acid sequence as set forth in SEQ ID NO:14, or having at least 80%, 85%, 90%, or 95% sequence identity thereto. In some embodiments, the anti-human PD-1 binding domain of the bispecific antibody of the present disclosure comprises: a heavy chain variable region (1) comprising an HCDR1 comprising the amino acid sequence set forth in SEQ ID NO: 1, an HCDR2 comprising the amino acid sequence set forth in SEQ ID NO:2, and an HCDR3 comprising the amino acid sequence set forth in SEQ ID NO:3, and (2) comprising the amino acid sequence as set forth in SEQ ID NO:4, or having at least 80%, 85%, 90%, or 95% sequence identity thereto; and a light chain variable region (1) comprising an LCDR1 comprising the amino acid sequence set forth in SEQ ID NO:11, an LCDR2 comprising the amino acid sequence set forth in SEQ ID NO:12, and an LCDR3 comprising the amino acid sequence set forth in SEQ ID NO:13, and (2) comprising the amino acid sequence as set forth in SEQ ID NO:14, or having at least 80%, 85%, 90%, or 95% sequence identity thereto.

In some embodiments, the anti-human TGFβR2 binding domain of the bispecific antibody of the present disclosure comprises (1) a heavy chain variable region comprising an HCDR1 comprising the amino acid sequence set forth in SEQ ID NO:6, an HCDR2 comprising the amino acid sequence set forth in SEQ ID NO:7, and an HCDR3 comprising the amino acid sequence set forth in SEQ ID NO:8, and (2) a light chain variable region comprising an LCDR1 comprising the amino acid sequence set forth in SEQ ID NO: 11, an LCDR2 comprising the amino acid sequence set forth in SEQ ID NO:12, and an LCDR3 comprising the amino acid sequence set forth in SEQ ID NO:13. In some embodiments, the anti-human TGFβR2 binding domain of the bispecific antibody of the present disclosure comprises (1) a heavy chain variable region comprising the amino acid sequence as set forth in SEQ ID NO:9, or having at least 80%, 85%, 90%, or 95% sequence identity thereto, and (2) a light chain variable region comprising the amino acid sequence as set forth in SEQ ID NO:14, or having at least 80%, 85%, 90%, or 95% sequence identity thereto. In some embodiments, the anti-human TGFβR2 binding domain of the bispecific antibody of the present disclosure comprises: a heavy chain variable region (1) comprising an HCDR1 comprising the amino acid sequence set forth in SEQ ID NO:6, an HCDR2 comprising the amino acid sequence set forth in SEQ ID NO:7, and an HCDR3 comprising the amino acid sequence set forth in SEQ ID NO:8, and (2) comprising the amino acid sequence as set forth in SEQ ID NO:9, or having at least 80%, 85%, 90%, or 95% sequence identity thereto; and a light chain variable region (1) comprising an LCDR1 comprising the amino acid sequence set forth in SEQ ID NO:11, an LCDR2 comprising the amino acid sequence set forth in SEQ ID NO:12, and an LCDR3 comprising the amino acid sequence set forth in SEQ ID NO:13, and (2) comprising the amino acid sequence as set forth in SEQ ID NO: 14, or having at least 80%, 85%, 90%, or 95% sequence identity thereto.

In some embodiments, the anti-human PD-1 binding domain of the bispecific antibody of the present disclosure comprises (1) a heavy chain variable region comprising an HCDR1 comprising the amino acid sequence set forth in SEQ ID NO:1, an HCDR2 comprising the amino acid sequence set forth in SEQ ID NO:2, and an HCDR3 comprising the amino acid sequence set forth in SEQ ID NO:3, and (2) a light chain variable region comprising an LCDR1 comprising the amino acid sequence set forth in SEQ ID NO:11, an LCDR2 comprising the amino acid sequence set forth in SEQ ID NO:12, and an LCDR3 comprising the amino acid sequence set forth in SEQ ID NO:13, and the anti-human TGFβR2 binding domain of the bispecific antibody comprises (1) a heavy chain variable region comprising an HCDR1 comprising the amino acid sequence set forth in SEQ ID NO:6, an HCDR2 comprising the amino acid sequence set forth in SEQ ID NO:7, and an HCDR3 comprising the amino acid sequence set forth in SEQ ID NO:8, and (2) a light chain variable region comprising an LCDR1 comprising the amino acid sequence set forth in SEQ ID NO:11, an LCDR2 comprising the amino acid sequence set forth in SEQ ID NO:12, and an LCDR3 comprising the amino acid sequence set forth in SEQ ID NO:13. In some embodiments, the anti-human PD-1 binding domain of the bispecific antibody of the present disclosure comprises (1) a heavy chain variable region comprising the amino acid sequence as set forth in SEQ ID NO:4, or having at least 80%, 85%, 90%, or 95% sequence identity thereto, and (2) a light chain variable region comprising the amino acid sequence as set forth in SEQ ID NO:14, or having at least 80%, 85%, 90%, or 95% sequence identity thereto, and the anti-human TGFβR2 binding domain of the bispecific antibody comprises (1) a heavy chain variable region comprising the amino acid sequence as set forth in SEQ ID NO:9, or having at least 80%, 85%, 90%, or 95% sequence identity thereto, and (2) a light chain variable region comprising the amino acid sequence as set forth in SEQ ID NO:14, or having at least 80%, 85%, 90%, or 95% sequence identity thereto. In some embodiments, the anti-human PD-1 binding domain of the bispecific antibody of the present disclosure comprises: a heavy chain variable region (1) comprising an HCDR1 comprising the amino acid sequence set forth in SEQ ID NO:1, an HCDR2 comprising the amino acid sequence set forth in SEQ ID NO:2, and an HCDR3 comprising the amino acid sequence set forth in SEQ ID NO:3, and (2) comprising the amino acid sequence as set forth in SEQ ID NO:4, or having at least 80%, 85%, 90%, or 95% sequence identity thereto; and a light chain variable region (1) comprising an LCDR1 comprising the amino acid sequence set forth in SEQ ID NO:11, an LCDR2 comprising the amino acid sequence set forth in SEQ ID NO:12, and an LCDR3 comprising the amino acid sequence set forth in SEQ ID NO: 13, and (2) comprising the amino acid sequence as set forth in SEQ ID NO:14, or having at least 80%, 85%, 90%, or 95% sequence identity thereto, and the anti-human TGFβR2 binding domain of the bispecific antibody comprises: a heavy chain variable region (1) comprising an HCDR1 comprising the amino acid sequence set forth in SEQ ID NO:6, an HCDR2 comprising the amino acid sequence set forth in SEQ ID NO:7, and an HCDR3 comprising the amino acid sequence set forth in SEQ ID NO:8, and (2) comprising the amino acid sequence as set forth in SEQ ID NO:9, or having at least 80%, 85%, 90%, or 95% sequence identity thereto; and a light chain variable region (1) comprising an LCDR1 comprising the amino acid sequence set forth in SEQ ID NO:11, an LCDR2 comprising the amino acid sequence set forth in SEQ ID NO:12, and an LCDR3 comprising the amino acid sequence set forth in SEQ ID NO:13, and (2) comprising the amino acid sequence as set forth in SEQ ID NO:14, or having at least 80%, 85%, 90%, or 95% sequence identity thereto.

In some embodiments, the bispecific antibody includes a human heavy chain and light chain constant region. In some embodiments, the heavy chain constant region comprises a CH1 domain and a hinge region. In some embodiments, the heavy chain constant region comprises a CH2 domain. In some embodiments, the heavy chain constant region comprises a CH3 domain. In some embodiments, the heavy chain constant region comprises CH1, CH2 and CH3 domains. If the heavy chain constant region includes substitutions, such substitutions modify the properties of the antibody (e.g., increase or decrease one or more of: Fc receptor binding, antibody glycosylation, the number of cysteine residues, effector cell function, or complement function). In some embodiments, the antibody is an IgG antibody. In some embodiments, the antibody is selected from the group consisting of IgG1, IgG2, IgG3, and IgG4.

Antibodies, such as ANTIBODY A, can be made, for example, by preparing and expressing synthetic genes that encode the recited amino acid sequences or by mutating human germline genes to provide a gene that encodes the recited amino acid sequences. Moreover, this antibody and other bispecific antibodies can be obtained, e.g., using one or more of the following methods.

Humanized antibodies can be generated by replacing sequences of the Fv variable region that are not directly involved in antigen binding with equivalent sequences from human Fv variable regions. General methods for generating humanized antibodies are provided by Morrison, S. L., Science, 229:1202-1207 (1985), by Oi et al., BioTechniques, 4:214 (1986), and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al., J. Mol. Biol., 227:776-798 (1992); Cook, G. P. et al., Immunol. Today, 16: 237-242 (1995); Chothia, D. et al., J. Mol. Bio. 227:799-817 (1992); and Tomlinson et al., EMBO J., 14:4628-4638 (1995). The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and U.S. Pat. No. 6,407,213; Tempest et al. (1991) Biotechnology 9:266-271. Still another method is termed “humaneering” and is described, for example, in U.S. 2005-008625.

Antibodies disclosed herein can include a human Fc region, e.g., a wild-type Fc region or an Fc region that includes one or more alterations. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005-0037000.

Provided herein are compositions comprising a mixture of a bispecific antibody and one or more acidic variants thereof, e.g., wherein the amount of acidic variant(s) is less than about 80%, 70%, 60%, 60%, 50%, 40%, 30%, 30%, 20%, 10%, 5% or 1%. Also provided are compositions comprising a bispecific antibody comprising at least one deamidation site, wherein the pH of the composition is from about 5.0 to about 6.5, such that, e.g., at least about 90% of the bispecific antibodies are not deamidated (i.e., less than about 10% of the antibodies are deamidated). In some embodiments, less than about 5%, 3%, 2% or 1% of the antibodies are deamidated. The pH may be from 5.0 to 6.0, such as 5.5 or 6.0. In some embodiments, the pH of the composition is 5.5, 5.6, 5.7, 5.8, 5.9, 6.0, 6.1, 6.2, 6.3, 6.4 or 6.5.

An “acidic variant” is a variant of a polypeptide of interest which is more acidic (e.g., as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e., the neutral amide side chain has been converted to a residue with an overall acidic character.

The term “mixture” as used herein in reference to a composition comprising a bispecific antibody means the presence of both the desired bispecific antibody and one or more acidic variants thereof. The acidic variants may comprise predominantly deamidated bispecific antibody, with minor amounts of other acidic variant(s).

In some embodiments, the binding affinity (K_{D}), on-rate (K_{D} on) and/or off-rate (K_{D} off) of the antibody that was mutated to eliminate deamidation is similar to that of the wild-type antibody, e.g., having a difference of less than about 5 fold, 2 fold, 1 fold (100%), 50%, 30%, 20%, 10%, 5%, 3%, 2% or 1%.

Bispecific antibodies of the disclosure can be prepared as full length antibodies or low molecular weight forms thereof (e.g., F(ab′)₂ bispecific antibodies, sc(Fv)₂ bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Methods of Producing Antibodies

Antibodies may be produced in, for example, bacterial or eukaryotic cells. Some antibodies can be produced in bacterial cells, e.g., E. coli cells. Antibodies can also be produced in eukaryotic cells such as transformed cell lines (e.g., CHO, 293E, COS). In addition, antibodies can be expressed in a yeast cell such as Pichia (see, e.g., Powers et al., J Immunol Methods. 251:123-35 (2001)), Hansenula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

Antibodies for use in methods described herein can be produced in mammalian cells. Exemplary mammalian host cells for expressing an antibody include Chinese Hamster Ovary (CHO) cells (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NSO myeloma cells and SP2 cells, and a cell from a transgenic animal, e.g., a transgenic mammal. For example, the cell is a mammary epithelial cell.

In an exemplary system for antibody expression, a recombinant expression vector(s) encoding the antibody heavy chains and the antibody light chains of a bispecific antibody (e.g., ANTIBODY A) is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly.

The antibodies of the present disclosure can be isolated from inside or outside (such as from the medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Antibody Pharmaceutical Compositions and Administration

A bispecific antibody described herein can be formulated as a pharmaceutical composition for administration to a subject, e.g., to treat a disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see, e.g., Berge, S. M., et al. (1977) J. Pharm. Sci. 66:1-19).

The bispecific antibody can be administered to a subject, e.g., a subject in need thereof, for example, a human subject, by intravenous injection or infusion (IV).

The bispecific antibody can be administered as a fixed dose, or in a mg/kg patient weight dose. The dose can also be chosen to reduce or avoid production of antibodies against the bispecific antibody. Dosage regimens are adjusted to provide the desired response, e.g., a therapeutic response or a combinatorial therapeutic effect. Generally, doses of the bispecific antibody can be used in order to provide a subject with the agent in bioavailable quantities.

For example, doses in the range of about 0.1 mg/kg to about 30 mg/kg can be administered. In some embodiments, a subject is administered the antibody at a dose of about 0.1 mg/kg to about 10 mg/kg (e.g., a dose of about 0.1 mg/kg, 0.5 mg/kg, 1 mg/kg, 1.5 mg/kg, 2 mg/kg, 3 mg/kg, 5 mg/kg, 6 mg/kg, 7.5 mg/kg, or about 10 mg/kg). In other embodiments, a subject is administered the antibody at a dose of about 1 mg/kg to about 3 mg/kg (e.g., a dose of about 1 mg/kg, 2 mg/kg, or 3 mg/kg). With respect to doses or dosages, the term “about” is intended to denote a range that is +10% of a recited dose, such that, for example, a dose of about 3 mg/kg will be between 2.7 mg/kg and 3.3 mg/kg patient weight.

Dosage unit form or “fixed dose” or “flat dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody may be administered via continuous infusion. For example, flat doses in the range of about 20 mg to 2500 mg can be administered. In some embodiments, a subject is administered the antibody at a dose of about 20 mg, 50 mg, 100 mg, 150 mg, 200 mg, 250 mg, 300 mg, 350 mg, 400 mg, 450 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, or 2500 mg. In some embodiments, a subject is administered the antibody at a dose of about 100 mg, 300 mg, 900 mg, 1500 mg, or 2000 mg.

A bispecific antibody dose can be administered, e.g., at a periodic interval over a period of time (a course of treatment) sufficient to encompass at least 2 doses, 3 doses, 5 doses, 10 doses, or more, e.g., weekly, biweekly (every two weeks), every three weeks, every four weeks, monthly, e.g., for between about 1 to 12 weeks. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments.

An exemplary flat dose dosing regimen comprises intravenous administration of a bispecific antibody described herein (e.g., ANTIBODY A) at a dosage of about 100 mg once every two weeks.

An further exemplary flat dose dosing regimen comprises intravenous administration of a bispecific antibody described herein (e.g., ANTIBODY A) at a dosage of about 300 mg once every two weeks.

An further exemplary flat dose dosing regimen comprises intravenous administration of a bispecific antibody described herein (e.g., ANTIBODY A) at a dosage of about 900 mg once every two weeks.

An further exemplary flat dose dosing regimen comprises intravenous administration of a bispecific antibody described herein (e.g., ANTIBODY A) at a dosage of about 1500 mg once every two weeks.

An further exemplary flat dose dosing regimen comprises intravenous administration of a bispecific antibody described herein (e.g., ANTIBODY A) at a dosage of about 2000 mg once every two weeks.

An exemplary flat dose dosing regimen comprises intravenous administration of a bispecific antibody described herein (e.g., ANTIBODY A) at a dosage of about 100 mg once every four weeks.

An further exemplary flat dose dosing regimen comprises intravenous administration of a bispecific antibody described herein (e.g., ANTIBODY A) at a dosage of about 300 mg once every four weeks.

An further exemplary flat dose dosing regimen comprises intravenous administration of a bispecific antibody described herein (e.g., ANTIBODY A) at a dosage of about 900 mg once every four weeks.

An further exemplary flat dose dosing regimen comprises intravenous administration of a bispecific antibody described herein (e.g., ANTIBODY A) at a dosage of about 1500 mg once every four weeks.

An further exemplary flat dose dosing regimen comprises intravenous administration of a bispecific antibody described herein (e.g., ANTIBODY A) at a dosage of about 2000 mg once every four weeks.

A pharmaceutical composition may include a “therapeutically effective amount” of a bispecific antibody described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

### Indications

A bispecific antibody described herein (e.g., ANTIBODY A) can be used to treat non-small cell lung cancer (NSCLC). In some embodiments, the NSCLC has squamous or nonsquamous histology. In some embodiments, the NSCLC is advanced or metastatic. In some embodiments, the subject has experienced disease progression after prior treatment (e.g., prior anti-PD-(L)1 therapy and/or anti-CTLA4 therapy).

A bispecific antibody described herein (e.g., ANTIBODY A) can be used to treat squamous cell carcinoma of the head and neck (SCCHN). In some embodiments, the SCCHN comprises a primary squamous tumor of the oral cavity, oropharynx, hypopharynx, or larynx. In some embodiments, the SCCHN comprises human papilloma virus (HPV)-positive SCCHN. In some embodiments, the SCCHN is advanced or metastatic. In some embodiments, the subject has experienced disease progression after prior treatment (e.g., prior anti-PD-(L)1 therapy and/or anti-CTLA4 therapy).

A bispecific antibody described herein (e.g., ANTIBODY A) can be used to treat cervical squamous cell carcinoma and endocervical adenocarcinoma (CESC). In some embodiments, the CESC comprises human papilloma virus (HPV)-positive CESC. In some embodiments, the CESC is advanced or metastatic. In some embodiments, the subject has experienced disease progression after prior treatment (e.g., prior anti-PD-(L)1 therapy and/or anti-CTLA4 therapy).

A bispecific antibody described herein (e.g., ANTIBODY A) can be used to treat ovarian cancer. In some embodiments, the ovarian cancer comprises ovarian epithelial carcinoma, fallopian tube carcinoma, primary peritoneal carcinoma, or carcinosarcoma. In some embodiments, the ovarian cancer is advanced or metastatic. In some embodiments, the subject has experienced disease progression after prior treatment (e.g., prior anti-PD-(L)1 therapy and/or anti-CTLA4 therapy).

A bispecific antibody described herein (e.g., ANTIBODY A) can be used to treat breast cancer. In some embodiments, the breast cancer comprises a triple negative breast cancer. In some embodiments, the triple negative breast cancer comprises HER2-negative, ER-negative, and PgR-negative breast cancer. In some embodiments, the breast cancer is advanced or metastatic. In some embodiments, the subject has experienced disease progression after prior treatment (e.g., prior anti-PD-(L)1 therapy and/or anti-CTLA4 therapy).

A bispecific antibody described herein (e.g., ANTIBODY A) can be used to treat bladder cancer. In some embodiments, the bladder cancer comprises urothelial carcinoma. In some embodiments, the bladder cancer is advanced or metastatic. In some embodiments, the subject has experienced disease progression after prior treatment (e.g., prior anti-PD-(L)1 therapy and/or anti-CTLA4 therapy).

A bispecific antibody described herein (e.g., ANTIBODY A) can be used to treat renal cell carcinoma. In some embodiments, the renal cell carcinoma is advanced or metastatic. In some embodiments, the subject has experienced disease progression after prior treatment (e.g., prior anti-PD-(L)1 therapy and/or anti-CTLA4 therapy).

A bispecific antibody described herein (e.g., ANTIBODY A) can be used to treat melanoma. In some embodiments, the melanoma is cutaneous malignant melanoma. In some embodiments, the melanoma is advanced or metastatic. In some embodiments, the subject has experienced disease progression after prior treatment (e.g., prior anti-PD-(L)1 therapy and/or anti-CTLA4 therapy).

A bispecific antibody described herein (e.g., ANTIBODY A) can be used to treat gastric adenocarcinoma. In some embodiments, gastric adenocarcinoma is advanced or metastatic. In some embodiments, the subject has experienced disease progression after prior treatment (e.g., prior anti-PD-(L)1 therapy and/or anti-CTLA4 therapy).

A bispecific antibody described herein (e.g., ANTIBODY A) can be used to treat esophageal cancer. In some embodiments, the esophageal cancer is advanced or metastatic. In some embodiments, the subject has experienced disease progression after prior treatment (e.g., prior anti-PD-(L)1 therapy and/or anti-CTLA4 therapy).

A bispecific antibody described herein (e.g., ANTIBODY A) can be used to treat gastroesophageal adenocarcinoma. In some embodiments, the gastroesophageal adenocarcinoma is advanced or metastatic. In some embodiments, the subject has experienced disease progression after prior treatment (e.g., prior anti-PD-(L)1 therapy and/or anti-CTLA4 therapy).

A bispecific antibody described herein (e.g., ANTIBODY A) can be used to treat malignant pleural mesothelioma. In some embodiments, the malignant pleural mesothelioma is advanced or metastatic. In some embodiments, the subject has experienced disease progression after prior treatment (e.g., prior anti-PD-(L)1 therapy and/or anti-CTLA4 therapy).

A bispecific antibody described herein (e.g., ANTIBODY A) can be used to treat pancreatic adenocarcinoma. In some embodiments, the pancreatic adenocarcinoma is advanced or metastatic. In some embodiments, the subject has experienced disease progression after prior treatment (e.g., prior anti-PD-(L)1 therapy and/or anti-CTLA4 therapy).

A bispecific antibody described herein (e.g., ANTIBODY A) can be used to treat colorectal cancer (CRC). In some embodiments, the CRC comprises microsatellite stable colorectal cancer (MSS-CRC). In some embodiments, the CRC comprises deficient mismatch repair (dMMR)/high microsatellite instability (MSI-H) colorectal cancer (dMMR/MSI-H CRC). In some embodiments, the CRC is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) for use in the treatment of non-small cell lung cancer (NSCLC). In some embodiments, the NSCLC has squamous or nonsquamous histology. In some embodiments, the NSCLC is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) for use in the treatment of squamous cell carcinoma of the head and neck (SCCHN). In some embodiments, the SCCHN comprises a primary squamous tumor of the oral cavity, oropharynx, hypopharynx, or larynx. In some embodiments, the SCCHN comprises human papilloma virus (HPV)-positive SCCHN. In some embodiments, the SCCHN is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) for use in the treatment of cervical squamous cell carcinoma and endocervical adenocarcinoma (CESC). In some embodiments, the CESC comprises human papilloma virus (HPV)-positive CESC. In some embodiments, the CESC is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) for use in the treatment of ovarian cancer. In some embodiments, the ovarian cancer comprises ovarian epithelial carcinoma, fallopian tube carcinoma, primary peritoneal carcinoma, or carcinosarcoma. In some embodiments, the ovarian cancer is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) for use in the treatment of breast cancer. In some embodiments, the breast cancer comprises a triple negative breast cancer. In some embodiments, the triple negative breast cancer comprises HER2-negative, ER-negative, and PgR-negative breast cancer. In some embodiments, the breast cancer is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) for use in the treatment of bladder cancer. In some embodiments, the bladder cancer comprises urothelial carcinoma. In some embodiments, the bladder cancer is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) for use in the treatment of renal cell carcinoma. In some embodiments, the renal cell carcinoma is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) for use in the treatment of melanoma. In some embodiments, the melanoma is cutaneous malignant melanoma. In some embodiments, the melanoma is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) for use in the treatment of gastric adenocarcinoma. In some embodiments, gastric adenocarcinoma is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) for use in the treatment of esophageal cancer. In some embodiments, the esophageal cancer is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) for use in the treatment of gastroesophageal adenocarcinoma. In some embodiments, the gastroesophageal adenocarcinoma is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) for use in the treatment of malignant pleural mesothelioma. In some embodiments, the malignant pleural mesothelioma is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) for use in the treatment of pancreatic adenocarcinoma. In some embodiments, the pancreatic adenocarcinoma is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) for use in the treatment of colorectal cancer (CRC). In some embodiments, the CRC comprises microsatellite stable colorectal cancer (MSS-CRC). In some embodiments, the CRC comprises deficient mismatch repair (dMMR)/high microsatellite instability (MSI-H) colorectal cancer (dMMR/MSI-H CRC). In some embodiments, the CRC is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) in the manufacture of a medicament for treating non-small cell lung cancer (NSCLC). In some embodiments, the NSCLC has squamous or nonsquamous histology. In some embodiments, the NSCLC is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) in the manufacture of a medicament for treating squamous cell carcinoma of the head and neck (SCCHN). In some embodiments, the SCCHN comprises a primary squamous tumor of the oral cavity, oropharynx, hypopharynx, or larynx. In some embodiments, the SCCHN comprises human papilloma virus (HPV)-positive SCCHN. In some embodiments, the SCCHN is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) in the manufacture of a medicament for treating cervical squamous cell carcinoma and endocervical adenocarcinoma (CESC). In some embodiments, the CESC comprises human papilloma virus (HPV)-positive CESC. In some embodiments, the CESC is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) in the manufacture of a medicament for treating ovarian cancer. In some embodiments, the ovarian cancer comprises ovarian epithelial carcinoma, fallopian tube carcinoma, primary peritoneal carcinoma, or carcinosarcoma. In some embodiments, the ovarian cancer is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) in the manufacture of a medicament for treating breast cancer. In some embodiments, the breast cancer comprises a triple negative breast cancer. In some embodiments, the triple negative breast cancer comprises HER2-negative, ER-negative, and PgR-negative breast cancer. In some embodiments, the breast cancer is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) in the manufacture of a medicament for treating bladder cancer. In some embodiments, the bladder cancer comprises urothelial carcinoma. In some embodiments, the bladder cancer is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) for use in the treatment of renal cell carcinoma. In some embodiments, the renal cell carcinoma is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) in the manufacture of a medicament for treating melanoma. In some embodiments, the melanoma is cutaneous malignant melanoma. In some embodiments, the melanoma is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) in the manufacture of a medicament for treating gastric adenocarcinoma. In some embodiments, gastric adenocarcinoma is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) in the manufacture of a medicament for treating esophageal cancer. In some embodiments, the esophageal cancer is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) in the manufacture of a medicament for treating gastroesophageal adenocarcinoma. In some embodiments, the gastroesophageal adenocarcinoma is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) in the manufacture of a medicament for treating malignant pleural mesothelioma. In some embodiments, the malignant pleural mesothelioma is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) in the manufacture of a medicament for treating pancreatic adenocarcinoma. In some embodiments, the pancreatic adenocarcinoma is advanced or metastatic. In some embodiments, the subject has experienced disease progression after prior treatment (e.g., prior anti-PD-(L)1 therapy and/or anti-CTLA4 therapy).

Another aspect comprises a bispecific antibody described herein (e.g., ANTIBODY A) in the manufacture of a medicament for treating colorectal cancer (CRC). In some embodiments, the CRC comprises microsatellite stable colorectal cancer (MSS-CRC). In some embodiments, the CRC comprises deficient mismatch repair (dMMR)/high microsatellite instability (MSI-H) colorectal cancer (dMMR/MSI-H CRC). In some embodiments, the CRC is advanced or metastatic. In some embodiments, the subject has experienced disease progression after prior treatment (e.g., prior anti-PD-(L)1 therapy and/or anti-CTLA4 therapy).

The following are examples of the practice of the invention. They are not to be construed as limiting the scope of the invention in any way.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

Reference antibodies and control antibodies used in the Examples include:
Reference PD-1 antibody pembrolizumab (also referred to as pembro or anti-PD-1) (made by Merck, distributed by Myonex);
Reference PD-L1 antibody atezolizumab (also referred to as atezo) (made by Genentech);
Reference antibody cetuximabx OKT3, which is a bispecific antibody comprising portions of cetuximab (anti-EGFR antibody) for targeting EGFR and OKT3 (anti-CD3 antibody) for targeting CD3;
Reference antibody NIS793 (made by Novartis), which is a fully human anti-TGFβ IgG2 monoclonal antibody designed to inhibit the TGFβ pathway;
Reference anti-TGFβR2 antibody TGF1 (also referred to as TGF1 or αTGFβR2 or anti-TGFBRII or anti-TGFBR2), which is a bivalent monospecific analog of TGF1 and comprises two heavy chains having an amino acid sequence as set forth in SEQ ID NO:16 and two light chains having an amino acid sequence as set forth in SEQ ID NO:17; and
Negative control IgG1 antibody (also referred to as RSV×RSV or RSV control or RSV or αRSV or aRSV or anti-RSV), which is a bivalent monospecific antibody comprising two heavy chains having an amino acid sequence as set forth in SEQ ID NO:18 and two light chains having an amino acid sequence as set forth in SEQ ID NO: 19.

TABLE 4
Reference Antibody Sequences

| Antibody | |
|---|---|
| Feature | Sequences |
| Reference | QLQVQESGPGLVKPSETLSLTCTVSGGSISNSYFS |
| TGFβR2 | WGWIRQPPGKGLEWIGSFYYGEKTYYNPSLKSRAT |
| antibody | ISIDTSKSQFSLKLSSVTAADTAVYYCPRGPTMIR |
| TGF1 | GVIDSWGQGTLVTVSSASTKGPSVFPLAPSSKSTS |
| HC | GGTAALGCLVKDYFPEPVTVSWNSGALTSGVHTFP |
| | AVLQSSGLYSLSSVVTVPSSSLGTQTYICNVNHKP |
| | SNTKVDKRVEPKSCDKTHTCPPCPAPELLGGPSVF |
| | LFPPKPKDTLMISRTPEVTCVVVDVSHEDPEVKFN |
| | WYVDGVEVHNAKTKPREEQYNSTYRVVSVLTVLHQ |
| | DWLNGKEYKCKVSNKALPAPIEKTISKAKGQPREP |
| | QVYTLPPSREEMTKNQVSLTCLVKGFYPSDIAVEW |
| | ESNGQPENNYKTTPPVLDSDGSFFLYSKLTVDKSR |
| | WQQGNVFSCSVMHEALHNHYTQKSLSLSPGK |
| | (SEQ ID NO: 16) |
| Reference | EIVLTQSPATLSLSPGERATLSCRASQSVRSYLAW |
| TGFβR2 | YQQKPGQAPRLLIYDASNRATGIPARFSGSGSGTD |
| antibody | FTLTISSLEPEDFAVYYCQQRSNWPPTFGQGTKVE |
| TGF1 | IKRTVAAPSVFIFPPSDEQLKSGTASVVCLLNNFY |
| LC | PREAKVQWKVDNALQSGNSQESVTEQDSKDSTYSL |
| | SSTLTLSKADYEKHKVYACEVTHQGLSSPVTKSFN |
| | RGEC (SEQ ID NO: 17) |
| Negative | EVQLVESGGGVVQPGRSLRLSCAASGFTFSNYGMH |
| control | WVRQAPGKGLEWVAVISYDGSTKYSADSLKGRFTI |
| IgG1 | SRDNSKNTLYLQMNSLRADDTAVYYCAKEGWSFDS |
| antibody | SGYRSWFDSWGQGTLVTVSSASTKGPSVFPLAPSS |
| (RSV) | KSTSGGTAALGCLVKDYFPEPVTVSWNSGALTSGV |
| HC | HTFPAVLQSSGLYSLSSVVTVPSSSLGTQTYICNV |
| | NHKPSNTKVDKRVEPKSCDKTHTCPPCPAPELLGG |
| | PSVFLFPPKPKDTLMISRTPEVTCVVVDVSHEDPE |
| | VKFNWYVDGVEVHNAKTKPREEQYNSTYRVVSVLT |
| | VLHQDWLNGKEYKCKVSNKALPAPIEKTISKAKGQ |
| | PREPQVYTLPPSREEMTKNQVSLTCLVKGFYPSDI |
| | AVEWESNGQPENNYKTTPPVLDSDGSFFLYSKLTV |
| | DKSRWQQGNVFSCSVMHEALHNHYTQKSLSLSPG |
| | (SEQ ID NO: 18) |
| Negative | DIQMTQSPSSLSASVGDRVTITCRASQSISSYLNW |
| control | YQQKPGKAPKLLIYAASSLQSGVPSRFSGSGSGTD |
| IgG1 | FTLTISSLQPEDFATYYCQQSYSTPPTFGQGTKVE |
| antibody | IKRTVAAPSVFIFPPSDEQLKSGTASVVCLLNNFY |
| (RSV) | PREAKVQWKVDNALQSGNSQESVTEQDSKDSTYSL |
| LC | SSTLTLSKADYEKHKVYACEVTHQGLSSPVTKSFN |
| | RGEC (SEQ ID NO: 19) |

### Example 1: Antibody A Overrode Immunosuppressive Effects of TGF-β on T Cell-Mediated Cytotoxicity in Renal Cancer Cells

This Example describes the in vitro assessment of various antibodies on the immunosuppressive effects of TGF-β on T cell-mediated cytotoxicity induced by a bispecific antibody against EGFR and CD3 (cetuximabxCD3).

In Vitro Cytotoxicity Assay: Adherent 786-O renal cancer cells were trypsinized (0.05% trypsin/EDTA; Gibco Cat #25300-054) and collected. Cells (1.0×10⁴ cells/well) were seeded in 96-well block-wall clear bottom plates (Greiner #655090) in complete phenol-free RPMI medium and incubated overnight at 37° C. in a humidified 5% CO₂ atmosphere. 786-O renal cancer cells were pre-incubated with bispecific antibodies or control antibodies in the absence or presence of TGF-β for 5-10 min at room temperature. Then, human CD3+ T cells in AIM V Medium were added in a 10:1 E/T ratio and incubated for 70 hours. Lactate dehydrogenase (LDH) release from membrane-damaged cells into supernatants was measured using a fluorometric method (560 nm Ex; 590 nm Em) according to the manufacturer's instructions (CytoTox-ONE™ Homogeneous Membrane Integrity Assay, Promega). Relative fluorescence units (RFU) in each treatment group and the fold changes in RFU over the control group were calculated.

Results: In order to mimic an immunosuppressive environment, exogenous TGF-β (0.1 ng/mL or 1.0 ng/mL) was added into the co-culture system of renal cancer cells and effector T cells. Cells were treated with cetuximabxCD3 bispecific antibody (BsAb) at 1.0 μg/ml in combination with one of the following antibodies: ANTIBODY A, anti-PD-1 antibody (pembrolizumab), anti-TGFβR2 antibody, or a bivalent antibody against RSV (RSV×RSV), which was used as a negative control antibody. Antibodies were added to cells at a concentration of 0.1 to 100 μg/mL.

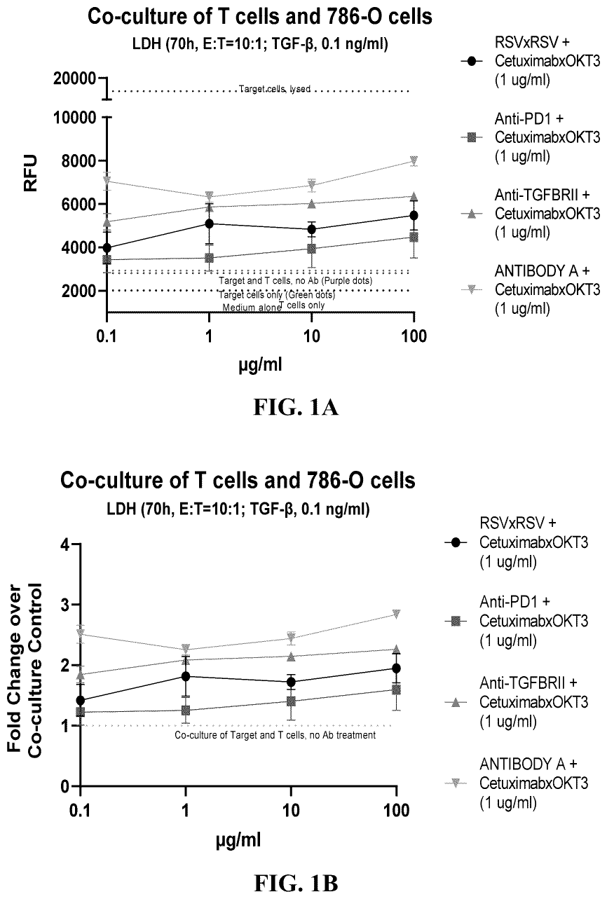

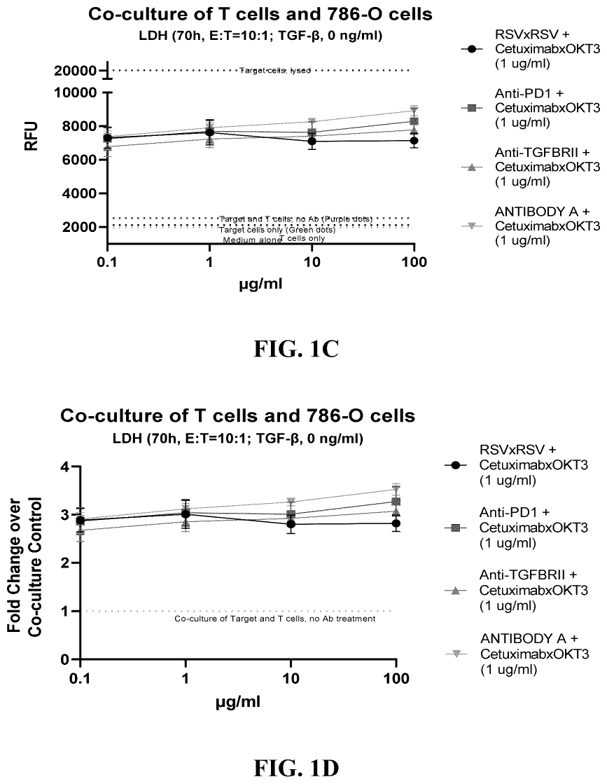

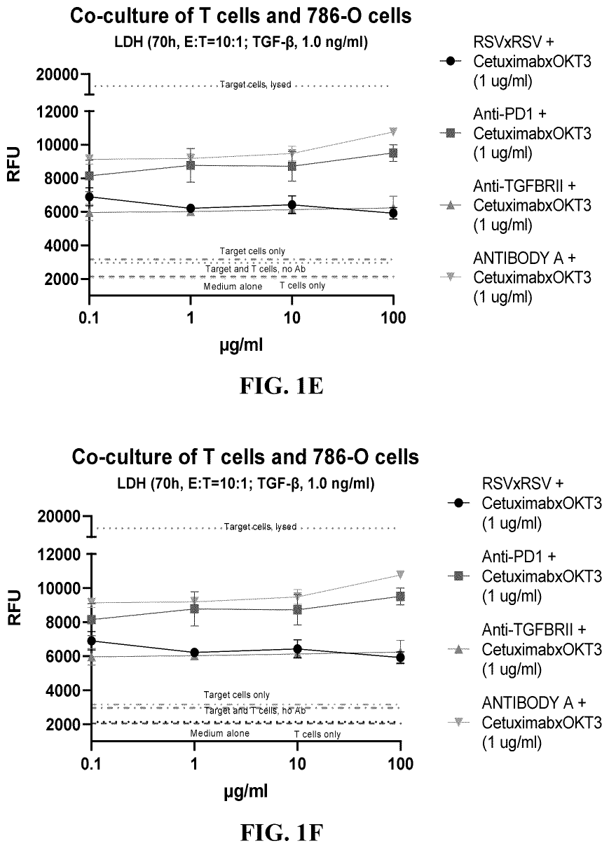

ANTIBODY A was more effective at counteracting the immunosuppressive effects of TGF-β (0.1 ng/mL) on T cell-mediated cytotoxicity induced by cetuximabxCD3 BsAb compared to the anti-PD-1 antibody, the anti-TGFβR2 antibody, or the RSV×RSV control antibody (FIGS. 1A-1B). ANTIBODY A produced similar effects in the co-culture system without TGF-β (FIGS. 1C-1D) or the co-culture system with TGF-β at a concentration of 1.0 ng/mL (FIGS. 1E-IF), albeit to a lesser extent than those produced in the co-culture system with TGF-β at a concentration of 0.1 ng/mL.

The concentration (1.0 μg/ml) of cetuximabxCD3 BsAb and the treatment time (70 hours) chosen for this combination study were based on the dose-response and time-course of cetuximabxCD3 BsAb alone inducing T-cell mediated target 786-O renal cancer cell killing.

### Example 2: Antibody a Inhibited Tumor Growth in a Xenograft Mouse Model of Melanoma

This Example describes the in vivo assessment of various antibodies on A375 human melanoma using a xenograft mouse model.

Antibodies and Formulation: ANTIBODY A was formulated in buffer containing 10 mM histidine, 263 mM sucrose, and pH 6.5. Compounds were solubilized in Dulbecco's phosphate-buffered saline (DPBS) (ThermoFisher Scientific, Waltham, MA; catalog #14190235). IgG1 control (Fc silent), TGF1 Fc silent (TGFβR2 antibody), and NIS793 (TGFβ1, 2, and 3 antibody) were produced by Incyte (Wilmington, DE). Atezolizumab (anti-PD-L1 antibody) and pembrolizumab (anti-PD-1 antibody) were purchased from RefDrug Inc. (Hillsborough Township, NJ).

A375 Xenograft Model: Female huCD34+ NSG mice (Jackson Labs, 19-21 weeks of age) were shaved, then inoculated subcutaneously with 5×10⁶ A375 cells (ATCC #CRL-1619) suspended in 200 μL DPBS (ThermoFisher Scientific) and Matrigel (Corning) mixed 1:1. On Day 8 post cell implantation, when tumors were approximately 160 mm³, mice were randomized by tumor size and stem cell donor into groups of 10. NSG mice engrafted with human CD34+ stem cells from 4 different donors were randomly allocated into each group of 10 mice (2-3 mice per donor within each group). Each group received 10 mg/kg of antibody in DPBS via intraperitoneal injection once every 5 days. Dose administration was continuous throughout the study. Plasma and tumor samples were collected from mice at 24, 72, or 108 hours after the last dose of antibody. Two-Way ANOVA was used to determine statistical differences between treatment groups.

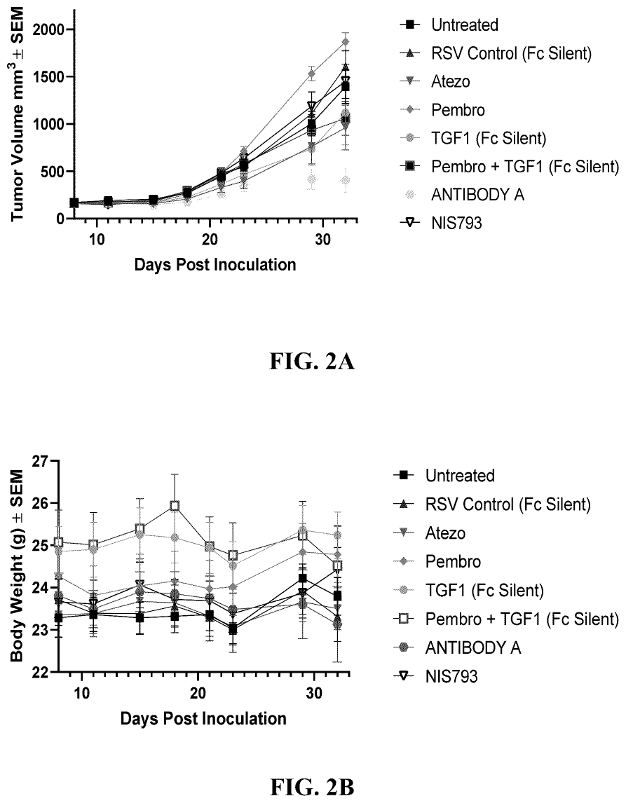

Results: To determine the in vivo efficacy of ANTIBODY A on A375 human melanoma, huCD34+ NSG mice were treated with either RSV×RSV control, atezolizumab, pembrolizumab, TGF1 (Fc Silent), a combination of pembrolizumab and TGF1, ANTIBODY A, or NIS793 at 10 mg/kg every 5 days. ANTIBODY A treatment resulted in statistically significant tumor growth inhibition (TGI) compared to RSV×RSV control antibody (TGI=75%, p=0.002) (FIG. 2A). The effect on tumor volume reduction in mice treated with ANTIBODY A was also statistically significant as compared to mice treated with the combination of pembrolizumab+TGF1 antibody (p<0.05), mice treated with pembrolizumab alone (p<0.05), and mice treated with TGF1 alone (p<0.0001) (FIG. 2A). The TGI values for all groups is shown in Table 5. There were no adverse effects on body weight of the animals with any of the treatments (FIG. 2B).

TABLE 5
TGI values

| Group | TGI |
|---|---|
| Atezolizumab | 40% |
| Pembrolizumab | −16% |
| TGF1 | 30% |
| Pembrolizumab + TGF1 | 34% |
| ANTIBODY A | 75% |
| NIS793 | 10% |

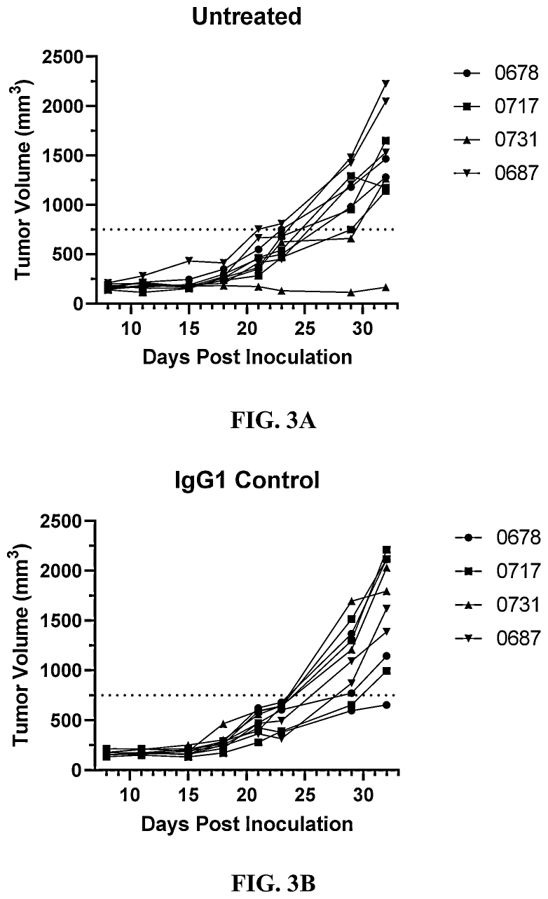

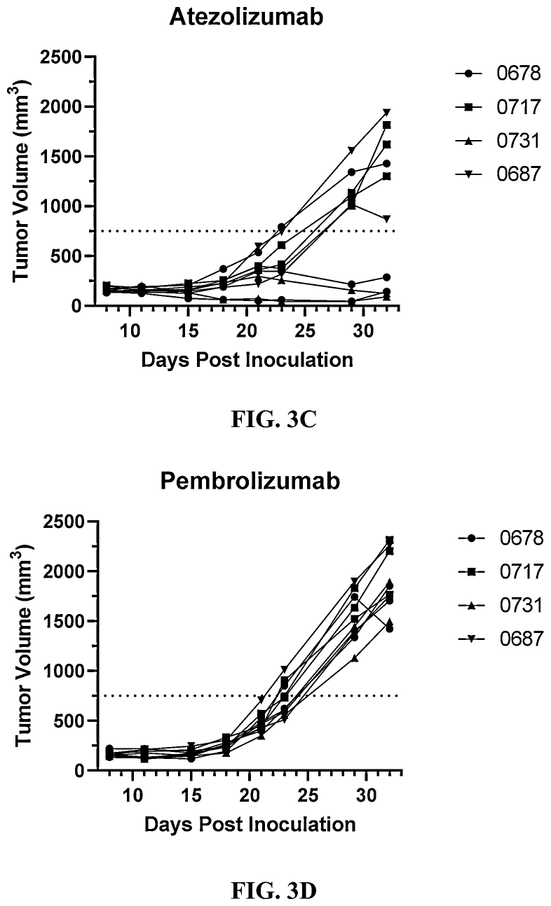

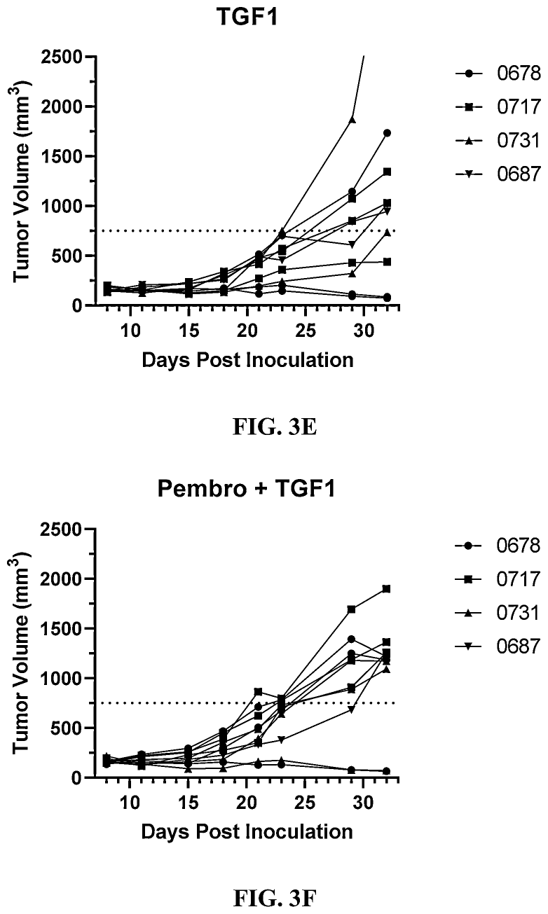

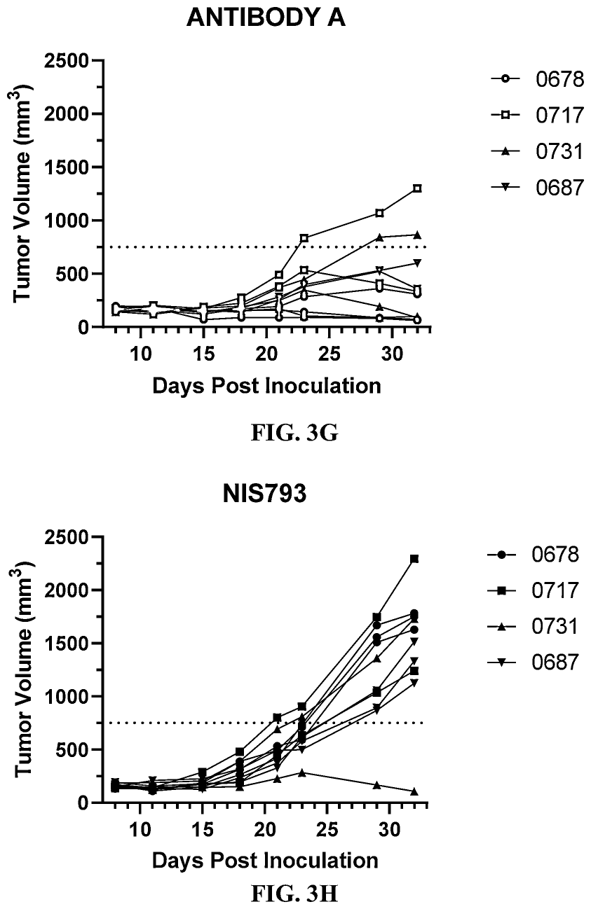

To determine the impact of stem cell donor on the activity of ANTIBODY A, individual tumor volumes for each treatment group were plotted according to stem cell donor. The data demonstrated that responses to ANTIBODY A were observed in all 4 donors (FIGS. 3A-3H). Partial responses were observed in mice receiving atezolizumab, TGF1, and pembrolizumab+TGF1.

### Example 3: Antibody A Inhibited Tumor Growth in a Xenograft Mouse Model of Triple-Negative Breast Cancer (TNBC)

This Example describes the in vivo assessment of various antibodies on MDA-MB-231 human triple-negative breast cancer (TNBC) using a xenograft mouse model.

Humanized NSG MDA-MB-231 Mouse Model: Humanized CD34 NSG mice were inoculated subcutaneously with a total of 3×10⁶ MDA-MB-231 tumor cells suspended in 100 μL of serum-free culture medium and matrigel matrix (Corning) in equal volumes. After tumors were established (80-100 mm³), the mice were randomized into the following treatment groups:
Negative control IgG1 antibody (RSV) (10 mg/kg);
ANTIBODY A (1 mg/kg)
ANTIBODY A (10 mg/kg); and
Pembrolizumab (10 mg/kg).

Each group had 8-9 mice. Animals were dosed intraperitoneally every five days for a period of 27 or 30 days. Tumors were measured using calipers, and the tumor volume was calculated by assimilating them to an ellipsoid using the formula: l (length)×w² (width)×½. Body weights were also monitored all through the study. Tumors were harvested (24 hours post last dosing) for tumor immune profiling and receptor occupancy post termination of the study.

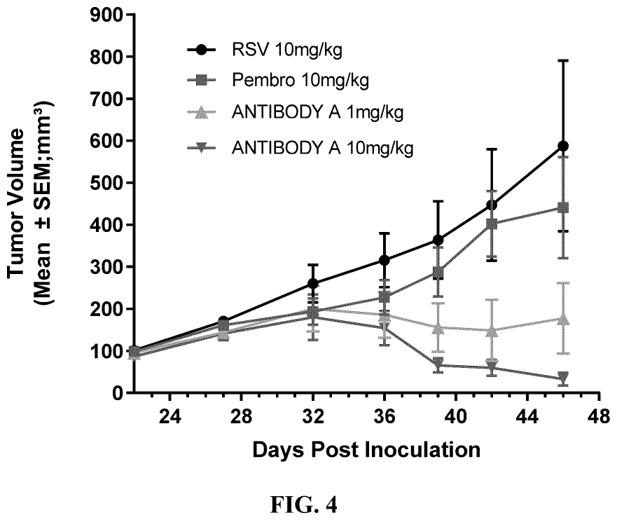

Results: The bispecific antibody, ANTIBODY A, induced a superior anti-tumor response compared to the reference PD-1 antibody pembrolizumab and the negative control IgG1 antibody RSV (FIG. 4). ANTIBODY A induced a superior anti-tumor response at both 1 mg/kg and 10 mg/kg dosage levels compared to pembrolizumab at 10 mg/kg (FIG. 4).

### Example 4: Antibody A Inhibited Tumor Growth in a Xenograft Mouse Model of Colorectal Cancer

This Example describes the in vivo assessment of various antibodies on MC38 human colorectal cancer using a xenograft mouse model.

Antibodies and Formulation: ANTIBODY A was formulated in buffer containing 10 mM histidine, 263 mM sucrose, and pH 6.5. Compounds were solubilized in Dulbecco's phosphate-buffered saline (DPBS) (ThermoFisher Scientific, Waltham, MA; catalog #14190235). Mice were treated with either a negative control IgG1 antibody (RSV), pembrolizumab (anti-PD-1 antibody), reference anti-TGFβR2 antibody (TGF1), pembrolizumab and reference anti-TGFβR2 antibody (TGF1), or ANTIBODY A.

MC38 Colorectal Cancer Xenograft Model: Female huCD34+ NSG mice (Jackson Labs, 19-21 weeks of age) were shaved, then inoculated subcutaneously with 2×10⁶ MC38 cells. On Day 9 post cell implantation, when tumors were approximately 110 mm³, mice were randomized by tumor size into groups of 9. Each group received 10 mg/kg of the indicated antibody via intraperitoneal injection. Mice treated with the combination of pembrolizumab and TGF1 received 10 mg/kg of each antibody. Antibody was administered twice a week. Cured mice were rechallenged with MC38 colorectal cancer cells and tumor volumes were monitored over time. Two-Way ANOVA was used to determine statistical differences between treatment groups.

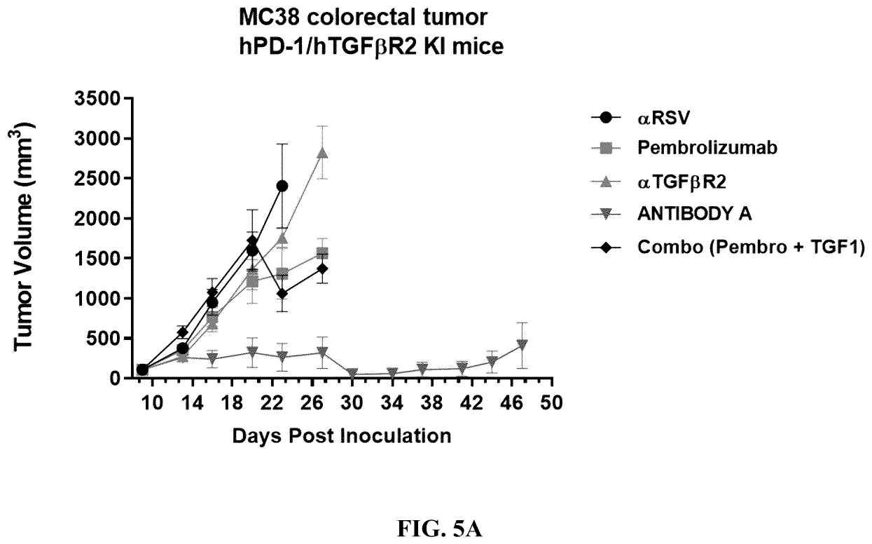

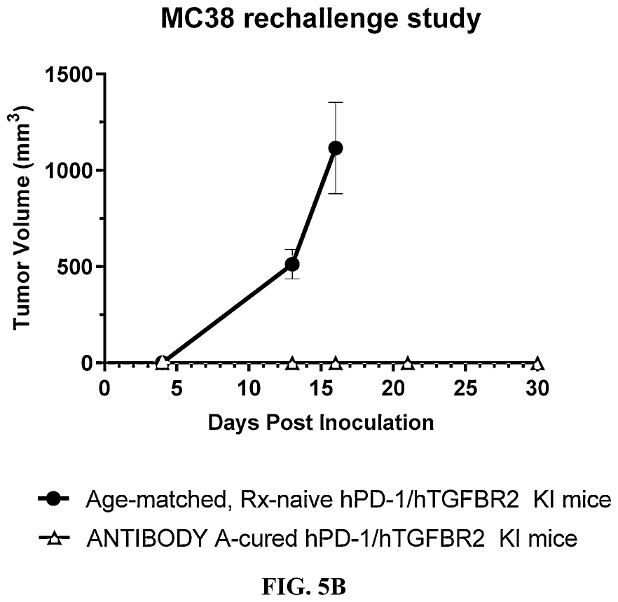

Results: Transgenic hPD-1/hTGFβR2 mice bearing MC38 colorectal tumors were treated intraperitoneally with various antibodies, and the tumor volumes were monitored over time. Both pembrolizumab and anti-TGFβR2 had minimal effect on reducing tumor volume, while ANTIBODY A significantly inhibited tumor growth and induced complete responses in some mice (FIG. 5A). ANTIBODY A also showed a superior effect on tumor volume compared to co-administration of an anti-TGFβR2 antibody (TGF1) and an anti-PD-1 antibody (pembrolizumab) (FIG. 5A). The ANTIBODY A-cured animals developed immunity against MC38 colorectal cancer reoccurrence (FIG. 5B).

### Example 5: Antibody A Increased Number of CD8 T Cells and Decreased Number of CD4 Tregs in MC38 Colorectal Tumors

This Example describes the flow cytometric analysis of MC38 colorectal tumors harvested from transgenic hPD-1/hTGFβR2 mice after treatment with various antibodies.

Flow Cytometry Analysis Of MC38 Colorectal Tumors: MC38 tumor cells (2 million cells) were inoculated into flanks of hPD-1/hTGFβR2 knock-in mice (Biocytogen). When tumors reached approximately 100 mm³, animals were randomized into different treatment groups, including anti-RSV, TGF1, pembrolizumab, a combination of pembrolizumab and TGF1, and ANTIBODY A. Antibodies were dosed at 10 mg/kg twice a week. ANTIBODY A was also dosed at 1 mg/kg twice a week. Tumors were harvested at 24 hours post the third dose, which was 8 days post first dose. Harvested MC38 tumors were cut into small fragments and digested using tumor dissociation kit (Miltenyi Biotec; cat #130-096-730) according to manufacturer's protocol. Samples were filtered through a 70 m filter (Corning, cat #352350) to form a single cell suspension for further analyses. Cells were blocked for 10 minutes with an anti-Fc receptor antibody (Biolegend, cat #101320), washed with 2% FBS in PBS, and then stained with fluorochrome-conjugated antibodies CD4 (BD Biosciences #624296), CD8 (eBioscience #365-0081-82), and CD25 (BD Biosciences #564368) for immunophenotyping analysis. Cell acquisition was performed under FACSymphony A3 (BD Biosciences) cytometer using DIVA software (BD Biosciences). Viability dye (Biolegend #423114) was included to exclude dead cells during analysis. Data analysis was performed using FlowJo software (BD Biosciences, version 10.8) and statistical analysis was done using GraphPad Prism (version 9.3.1).

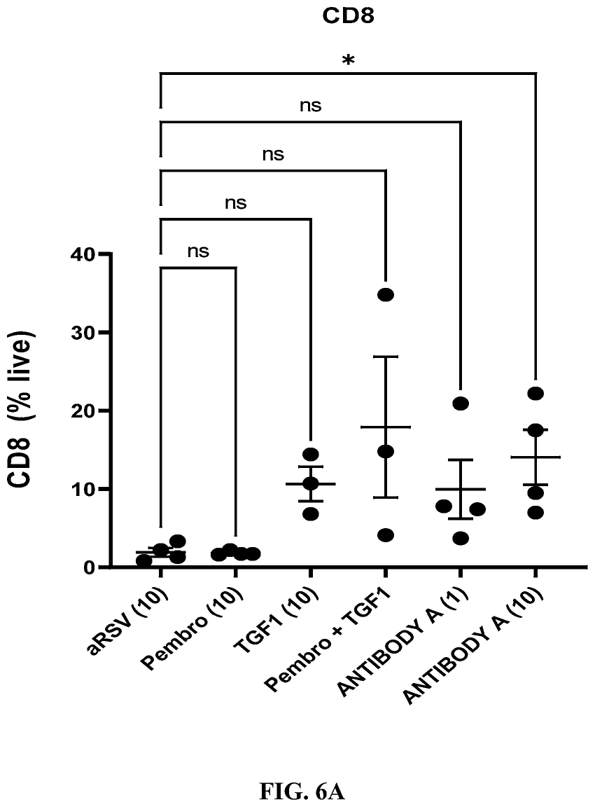

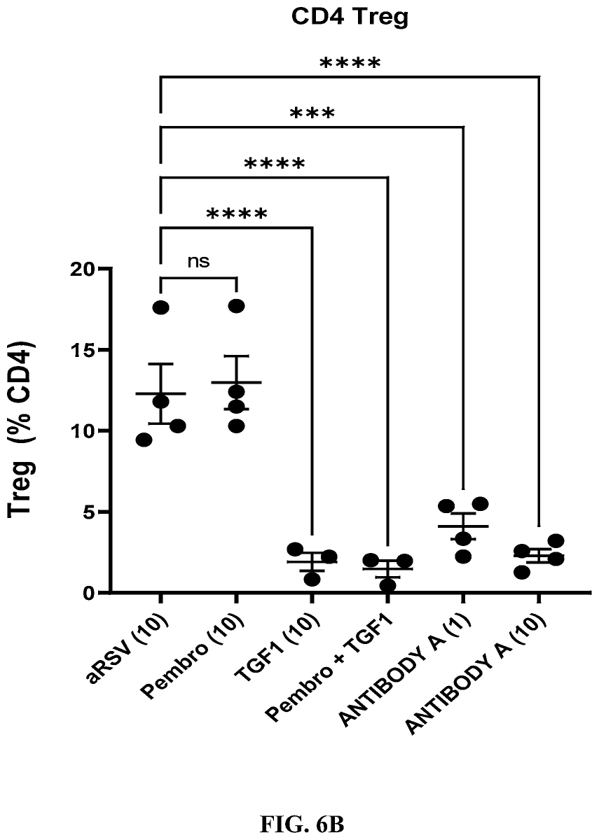

Results: MC38 colorectal tumors harvested from mice treated with ANTIBODY A at a dose of 10 mg/kg twice per week showed a statistically significant increase in number of CD8 T cells in the tumor compared to MC38 colorectal tumors harvested from mice treated with the RSV control antibody (FIG. 6A). Also, compared to tumors from RSV-treated mice, a statistically significant decrease in number of CD4 Tregs was observed in MC38 colorectal tumors harvested from mice treated with an anti-TGFβR2 antibody (TGF1), a combination of TGF1 and pembro, or ANTIBODY A at either the 1 mg/kg or 10 mg/kg dose (FIG. 6B). No change in the number of T cells and Tregs was observed in MC38 colorectal tumors harvested from mice treated with pembrolizumab alone compared to mice treated with the negative control antibody anti-RSV (FIGS. 6A-6B).

### Example 6: Antibody A Increased Number of CD8+ T Cells in MC38 Colorectal Tumors

This Example describes the immunohistochemistry (IHC) analysis of MC38 colorectal tumors harvested from transgenic hPD-1/hTGFβR2 mice after treatment with various antibodies.

IHC Analysis Of MC38 Colorectal Tumors: MC3 8 tumors were grown in hPD-1/hTGFBR2 knock-in mice and the mice were treated with RSV control antibody, ANTIBODY A (1 mg/kg), or ANTIBODY A (10 mg/kg) as described in Examples 4-5. Tumors were collected 24 hours post the third dose, fixed in 10% formalin overnight and embedded in paraffin blocks. For immunohistochemistry, 5 m sections were cut, mounted onto charged slides and air-dried overnight before staining. Anti-CD8a antibody (clone D4W2Z, 1:400, Cell Signaling Technology, Cat. #98941) was used to stain T lymphocytes. Stained slides were digitally scanned and analyzed. Statistical analyses were performed using one way ANOVA in GraphPad PRISM (version 9.3.1). All data were presented as Mean SD. **p<0.01.

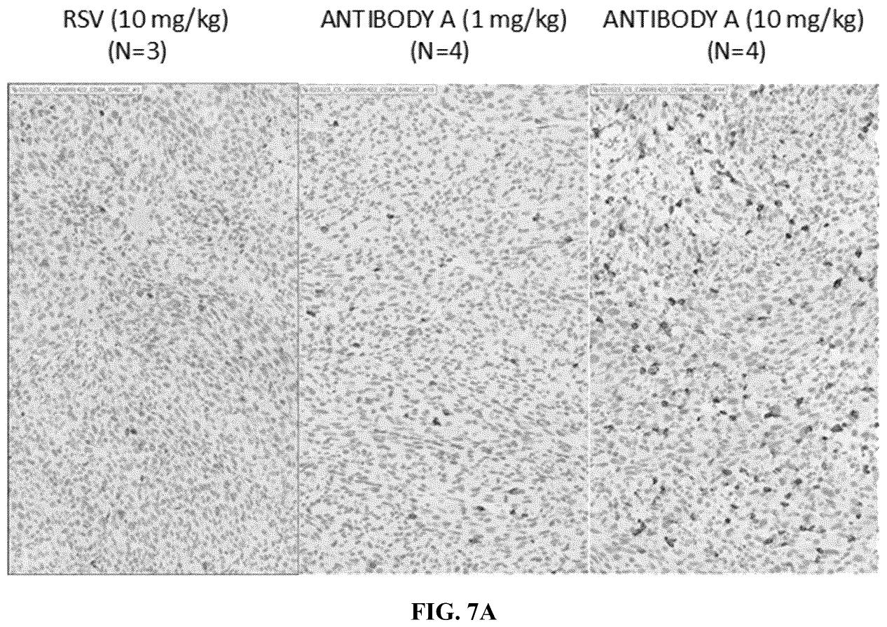

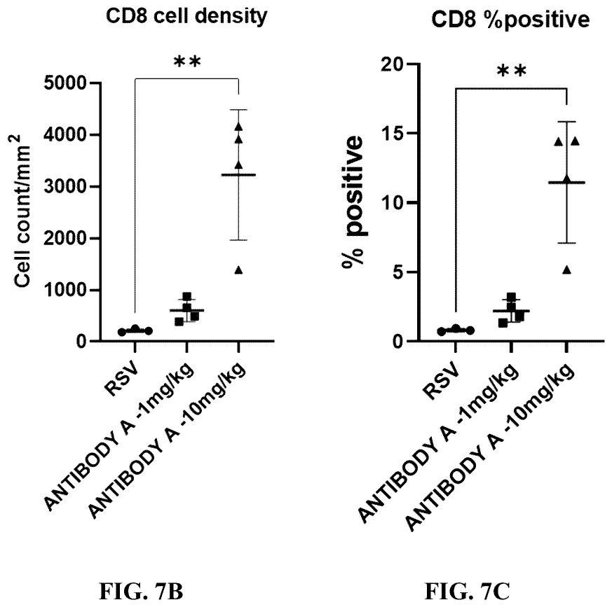

Results: Representative photomicrographs are shown in FIG. 7A (left panel: RSV control; middle panel: ANTIBODY A 1 mg/kg; right panel: Antibody A 10 mg/kg). Graphs showing the CD8 cell density and percent CD8 positive cells in the control and treatment groups are shown in FIG. 7B and FIG. 7C, respectively. Taken together, these results demonstrated that tumors treated with ANTIBODY A showed a dose-dependent CD8+ T cell infiltration, which is increased as compared to the RSV control.

### Example 7: Antibody A Induced IFNγ Production in Ex Vivo Samples from Non-Small Cell Lung Cancer (NSCLC) Patients

This Example describes IFNγ production in tumor digests from NSCLC patients in the presence of various antibodies.

Study Design: A total of 10 patients with Stage I-II lung cancer, who were scheduled for surgical resection, consented for collection of a portion of their tumor tissue and/or blood for research purposes after obtaining consent that had been approved by Institutional Review Board. All patients selected for entry into the study met the following criteria: (i) histologically confirmed pulmonary squamous cell carcinoma (SCC) or adenocarcinoma (AC), (ii) no prior chemotherapy or radiation therapy within two years, and (iii) no other active malignancy.

Reagents: The enzymatic cocktail for tumor digestion consisted of serum-free Hyclone™ Leibovitz L-15 media supplemented with 1% Penicillin-Streptomycin, Collagenase type I and IV (170 mg/L=45-60 U/mL), Collagenase type II (56 mg/L=15-20 U/mL), DNase-I (25 mg/L), and Elastase (25 mg/L) (all from Worthington Biochemical, NJ). The culture media DMEM/F-12 1:1 (HyClone, Thermo Scientific) was supplemented with 2.5 mM L-glutamine, 15 mM HEPES Buffer, 10% of Embryonic Stem (ES) Cell Screened FBS (U.S.) (Thermo Scientific™ HyClone™), Penicillin (100 U/ml) and Streptomycin (100 μg/mL), hereafter referred to as complete cell culture media.

Preparation Of A Single-Cell Suspension From Tumor Lung Tissue: Surgically-resected fresh lung tumors were processed within 20 minutes of removal from the patient. An optimized disaggregation method for human lung tumors that preserves the phenotype and function of the immune cells was used. Briefly, under sterile conditions, all areas of tissue necrosis were trimmed away. The tumor lung tissues were sliced into 1-2 mm³ pieces with micro-dissecting scissors equipped with tungsten carbide insert blades. For enzymatic digestion, the pieces were incubated in a shaker for 45 minutes at 37° C. in serum-free L-15 Leibovitz media (HyClone) containing enzymes and 1% Penicillin-Streptomycin (Life Technologies, Carlsbad, CA). L-15 Leibovitz media was formulated for use in carbon dioxide-free systems. After 45 minutes, any visible tumor pieces were vigorously pipetted against the side of a 50 mL tube to enhance disaggregation and then further incubated for 30-50 minutes under the same conditions. Larger pieces of tumor tissue were permitted to settle to the bottom of the tube and the supernatant was passed through a 70 μM nylon cell strainer (BD Falcon). The remaining pieces in the tube underwent further pipetting before being passed through the same cell strainer. After filtration, the red blood cells were lysed using 1× Red Blood Cell (RBC) Lysis Buffer (Santa Cruz, Dallas, TX). The remaining cells were washed twice in RPMI supplemented with 2% FBS and re-suspended in the cell culture media. Cell viability, as determined by trypan blue exclusion or Fixable Viability Dye eFluor® 450 staining, was typically >90%. If the viability of cells was less than 80%, dead cells were eliminated using a “dead cell removal kit” (Miltenyi Biotec Inc., Germany).

Flow Cytometry: Flow cytometric analysis was performed according to standard protocols. Matched isotype antibodies were used as controls. Negative gating was based on fluorescence minus one (FMO) strategy. To exclude dead cells from analysis, cells were stained with the LIVE/DEAD® fixable dead cell stains (Molecular probes, Life Technologies).

For intracellular staining, fixed cells stained for surface markers were permeabilized with BD Perm/Wash™ Buffer (BD Biosciences) and then stained with FITC anti-human IFN-γ (Biolegend, clone: 4S.B3) for 45 minutes at RT. All data were acquired using the BD LSRFortessa™ (BD Bioscience) flow cytometers or CytoFLEX S (Beckman Coulter) and analyzed using FlowJo software (TreeStar Inc.).

Generation Of NY-ESO-1-Specific Ly95 T Cells And A549/A2-NY-ESO-1 Target Lung Cancer CellLine: The NY-ESO-1-reactive Ly95 TCR construct is an affinity-enhanced variant of the wild-type IG4 TCR identified from T cells recognizing the HLA-A2 restricted NY-ESO-1:157-165 peptide antigen. The generation of this Ly95 TCR construct and its packaging into a lentiviral vector was performed as previously described (Moon E K, et al. Blockade of Programmed Death 1 Augments the Ability of Human T Cells Engineered to Target NY-ESO-1 to Control Tumor Growth after Adoptive Transfer. C/in Cancer Res. 2016; 22(2):436-47). Human T cells were isolated from PBMC of healthy volunteer donors by negative selection using RosetteSep kits (Stem Cell Technologies, Vancouver, Canada). Isolated T cells were stimulated with magnetic beads coated with anti-CD3/anti-CD28 at a 1:3 cell to bead ratio. T cells were transduced with lentiviral vectors at a multiplicity of infection (MOI) of approximately 5. Cells were counted and fed with complete cell culture medium every 2 days. A small portion of expanded cells was stained for flow cytometry confirmation of successful Ly95 transduction using the VP 13.1 TCR chain antibody (Beckman Coulter: clone IMMU 222). Transduction of human T cells undergoing anti-CD3/CD28 mAb-coated bead activation with high titer lentivirus that encodes the Ly95 TCR recognizing NY-ESO-1 resulted in approximately 50% of TCRVb13.1⁺ CD8⁺ cells.

For target cells, the A549 human lung adenocarcinoma cell line was genetically modified to express both NY-ESO-1 protein and HLA-A*02 as described earlier (Moon E K, et al. Blockade of Programmed Death 1 Augments the Ability of Human T Cells Engineered to Target NY-ESO-1 to Control Tumor Growth after Adoptive Transfer. Clin Cancer Res. 2016; 22(2):436-47). Briefly, the A549 cell line was transduced by a retroviral vector encoding NY-ESO-1-T2A-HLA-A*02. The transduced A549 cells were subjected to limiting dilution at 0.5 cell per well in 96-well plates. The resulting clones were tested by flow cytometry for HLA-A*02 expression using anti-HLA-A2 Abs (Biolegend, clone: bb7.2). HLA-A2 positive clones were selected and tested in co-culture with T cells expressing the NY-ESO-1 Ly95 TCR. The clones expressing HLA-A2 that could stimulate NY-ESO-1 Ly95 TCR-expressing T cells to secrete IFN-γ were pooled to generate the A549-NY-ESO-1-A2 (A549-A2-ESO) cell line. Flow sorting was performed to enrich for high HLA-A2 expressing tumor cells. The expression of intracellular NY-ESO was analyzed by flow cytometry using NY-ESO-1 (D1Q2U) mAb (Cell Signaling) that recognizes endogenous levels of total NY-ESO-1 protein.

NY-ESO-Specific T Cell Response: TCR transduced T cells (Ly95 T cells) recognizing the HLA-A2 restricted NY-ESO-1:157-165 peptide antigen were used to study the regulation of antigen-specific effector T cell responses by tumor digests in the presence of ANTIBODY A. The A549 human lung adenocarcinoma cell line that was genetically modified to express both NY-ESO-1 protein and HLA-A*02 A549 (A2-NY-ESO-1 tumor cells) was used to stimulate the Ly95 T cell response. To evaluate the effects of lung tumors on antigen specific T cell responses, Ly95 cells were co-cultured with A549/A2-NY-ESO-1 tumor cells in the presence or absence of the single cell suspension obtained from digested tumors at 1:1:3 ratio (Ly95:A549:tumor) for 48 hours. ANTIBODY A or a control antibody at a concentration of 20 μg/mL was present in cell co-cultures for 48 hours, starting from the beginning of the assay. Matched isotype antibodies (20 μg/mL) were used as controls. To evaluate the effects of lung tumors on NY-ESO specific Ly95 T cell responses, IFN-γ production by Ly95 T cells was measured. Ly95 T cells at a concentration of 1.5×10⁵ cells/well (24 well plate) were mixed with A549 A2-NY-ESO-1 tumor cells in the presence of tumor digests and either ANTIBODY A or a control antibody. BD GolgiStop™ and BD GolgiPlug™ were added into the cell cultures during the last 6 hr. Ly95 T cells co-cultured with NY-ESO-1 negative A549 tumor cells were used as a negative control to define the level of allostimulation. Cells were collected, washed in Stain Buffer (BD Biosciences) and stained for CD8 and Ly95 TCR surface markers using anti-CD8 (Biolegend, clone: HIT8a) and anti-TCRVβ13.1 (Beckman Coulter: clone IMMU 222) antibodies and then stained for intracellular IFN-γ. Surface-stained cells were fixed with BD Cytofix™ Fixation Buffer (BD Biosciences) for 20 minutes. The fixed cells were permeabilized with BD Perm/Wash™ Buffer (BD Biosciences) and then stained with the anti-human IFN-γ (Biolegend, clone: 4S.B3). The production of IFN-γ was analyzed in gated live CD8⁺TCRVβ13.1⁺ cells by flow cytometry.

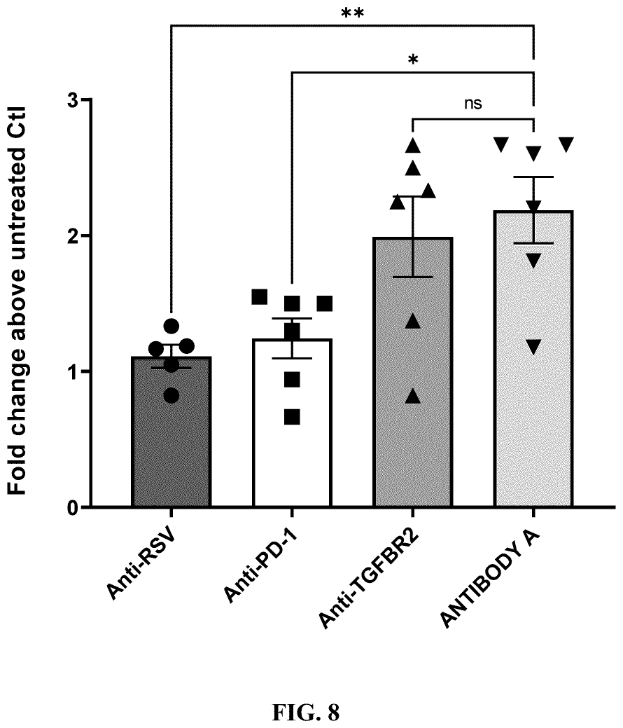

Results: Samples treated with ANTIBODY A showed a significant increase in percent IFNγ-producing Ly95 T cells compared to samples treated with the negative control antibody RSV or the anti-PD-1 antibody pembrolizumab (Table 6 and FIG. 8). These results demonstrate that ANTIBODY A can reactivate T cells to produce cytokines in ex vivo samples from NSCLC patients.

TABLE 6
Percent IFNγ producing CD8 cells analyzed by flow cytometry

| Treatment | % IFNγ producing CD8 cells |
|---|---|
| Untreated control | 2.7% |
| Anti-TGFβR2 | 9.1% |
| Pembrolizumab | 5.2% |
| ANTIBODY A | 15.0% |

### Example 8: Antibody A Induced IFNγ Production in Ex Vivo Samples from Ovarian Cancer Patients

This Example describes IFNγ production in tumor digests from ovarian cancer patients in the presence of various antibodies.

Methods: Frozen ascites samples of high-grade serous ovarian cancer patients were obtained from the tumor bank. Samples were thawed, cells were washed and tested for viability using an acridine orange/propidium iodide (AOPI) stain. Approximately 2×10⁶ viable cells were plated in 6 well plates in triplicates for all conditions indicated in the data. Drugs were added to appropriate wells at 10 mg/mL final concentration to the plated cells. Plates were incubated at 37° C. for 4 days. At the end of the incubation time period, media supernatant was collected from each well and diluted (1:5) for IFN-7 analysis. IFN-7 ELISA was performed using a commercially available kit (Biolegend #430104), following manufacturer's guidelines. Concentration of IFN-7 was calculated using the standard curve. The values were corrected for the dilution factor and plotted using GraphPad Prism. Statistical analyses were done using one-way ANOVA with multiple comparisons.

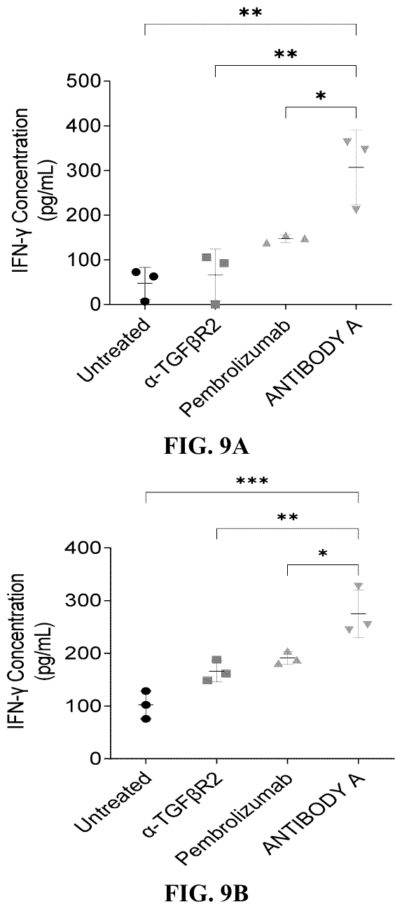

Results: ANTIBODY A significantly increased induction of IFNγ in ascites samples from ovarian cancer patients compared to untreated samples (FIGS. 9A-9B). The amount of induced IFNγ was greater in samples treated with ANTIBODY A compared to those treated with the negative control antibody RSV or the reference antibody to TGFβR2 (anti-TGFβR2) or the reference antibody to PD-1 (pembrolizumab) (FIGS. 9A-9B). These results demonstrate that ANTIBODY A can reactivate T cells to produce cytokines in ex vivo samples from ovarian cancer patients.

### Example 9: A Study of Antibody A in Participants with Selected Advanced Malignancies

This Example describes a phase 1, multicenter, open-label, dose-escalation, and dose-expansion clinical study to investigate the safety, tolerability, pharmacokinetic (PK), pharmacodynamics, and preliminary clinical efficacy of ANTIBODY A in participants with selected advanced malignancies.

Part 1 of the study is a dose escalation in participants with select advanced malignancies. Part 1 will assess the safety and tolerability and identify the maximum tolerated dose (MTD) and/or recommended dose for expansion (RDE). The select advanced malignancies included in Part 1 are non-small cell lung cancer (NSCLC), squamous cell carcinoma of the head and neck (SCCHN), cervical squamous cell carcinoma and endocervical adenocarcinoma (CESC), ovarian cancer, a breast cancer, bladder cancer, renal cell carcinoma, melanoma, gastric adenocarcinoma, esophageal cancer, gastroesophageal adenocarcinoma, malignant pleural mesothelioma, pancreatic adenocarcinoma, and colorectal cancer (CRC).

Part 2 of the study is an open-label dose expansion to further evaluate the safety, tolerability, PK, pharmacodynamics, and preliminary antitumor activity of ANTIBODY A at selected recommended dose(s) for expansion (RDE(s)) in two tumor-specific cohorts:

Cohort 1, ICI-Sensitive: Participants with the following selected advanced or metastatic solid tumors who have progressed on, are intolerant to, or are ineligible for standard of care therapies including immune checkpoint inhibitors (ICIs): bladder cancer, cervical squamous cell carcinoma and endocervical adenocarcinoma, esophageal cancer, gastric adenocarcinoma, gastroesophageal junction cancer, melanoma, malignant pleural mesothelioma, non-small cell lung cancer, ovarian cancer, renal cell carcinoma, squamous cell carcinoma of the head and neck, triple-negative breast cancer, or deficient mismatch repair/high microsatellite instability colorectal cancer.

Cohort 2, ICI-Nonsensitive: Participants with the following selected advanced or metastatic solid tumors who have progressed on, are intolerant to, or are ineligible for standard of care therapies (ICIs are not indicated): pancreatic adenocarcinoma or microsatellite stable colorectal cancer.

For Part 2, prior treatment with “standard therapy” includes available standard therapies, including anti-PD-(L)1 therapy and anti-CTLA-4 therapy, that are known to confer clinical benefit.

### Justification for Dose

For Part 1, an ANTIBODY A starting dose of 100 mg once every 2 weeks (Q2W) was selected since this dose is predicted to result in serum antibody levels that exceed the EC90 for ANTIBODY A binding to PD-1/TGFβR2-positive circulating T cells throughout the treatment period that results in conditional inhibition of TGFβ signaling in a PD-1-dependent manner.

The proposed safe starting dose (SSD) attempts to minimize the exposure of patients with advanced cancer to subtherapeutic dose levels of ANTIBODY A while balancing the safety risk associated with the nonclinical pharmacologic and toxicological profiles. Based on this assessment, the SSD is expected to be safe for the following reasons:

(1) In cynomolgus monkeys, the binding affinity of ANTIBODY A is similar to that in humans (9 nM and 0.6 nM for TGFβR2 and PD-1, respectively).

(2) Serum ANTIBODY A levels at all doses in the Good Laboratory Practice (GLP) toxicology study exceeded the EC₉₀ for ANTIBODY A binding in PD-1/TGFβR2-positive circulating T cells throughout the treatment period, indicating that the sustained inhibition of TGFβR2/PD-1 is not associated with significant toxicity.

(3) There is an absence of evidence for cytokine release that would be predictive of cytokine release syndrome in an in vitro whole blood cytokine release assay and no significant cytokine release in cynomolgus monkeys following administration of ANTIBODY A.

(4) There was no unexpected tissue staining in human tissues in a tissue cross-reactivity study.

(5) The high dose in the GLP toxicology study (75 mg/kg twice weekly) was determined to be the no-observed-adverse-effect level based on the absence of target organ toxicity, infusion reactions, cytokine release, and changes in immune cell populations.

(6) ANTIBODY A showed no to weak activity to inhibit TGFβ signaling in human aortic smooth muscle cells (HASMC) (concentrations up to 100 μg/mL). Consistent with the tissue cross-reactivity assay, ANTIBODY A was shown to bind to mononuclear leukocytes and not to other cell types that do not co-express PD-1, such as endothelial cells or cardiomyocytes.

(7) The SSD (100 mg Q2W) is below the maximum SSD of 240 mg/dose allowed under the ICH S9 guideline (ICH 2009) for determining the SSD for oncology agents.

(8) The predicted human exposure to ANTIBODY A at 100 mg Q2W is several orders of magnitude (235×) below the serum concentration at the no-observed-adverse-effect level dose in the GLP toxicology study (75 mg/kg; the highest dose on study).

(9) The SSD of 100 mg Q2W is projected to cover the ANTIBODY A in vitro mixed lymphocyte reaction (MLR) assay EC₅₀ around the clock and EC₉₀ for approximately 60 hours but not reach the in vitro pSMAD inhibition EC₅₀ (>100 μg/mL) for TGFβR2 single positive cells. Based on quantitative systems pharmacology (QSP) modeling and simulation, the predicted TGFβR2 target occupancy in a tumor at steady state is 30% at Cₘₐₓ and 17% at C_{trough} with a 100 mg Q2W dose regimen. Thus, the proposed SSD is expected to produce pharmacological activity without safety concern.

(10) ANTIBODY A 100 mg Q2W is 20-fold lower than the QSP-modeled projected safe dose, which is at least 2000 mg Q2W.

Based on preclinical data (e.g., toxicology, pharmacology, and PK data) as well as predicted human PK, ANTIBODY A 100 mg Q2W IV has been selected as the SSD for this Phase 1 study. The administration schedule of ANTIBODY A may be changed to Q4W based on emerging PK and pharmacodynamic data.

In Part 2, ANTIBODY A will be administered at the RDE(s) identified in Part 1. If more than 1 RDE is selected, the following criteria can be met: (1) RDEs do not have overlapping PK exposure (e.g., 2- to 3-fold apart); (2) RDE_{low} should not be lower than the minimal dose identified that exhibits pharmacological activity; and (3) RDE_{high} should not exceed the MTD. In the event that two RDEs are selected for evaluation within a particular dose-expansion cohort, participants will be randomized to receive one of the RDEs during study participation.

### Antibody A Regimen

The starting dose of ANTIBODY A in Part 1 is 100 mg administered by intravenous infusion Q2W. The following additional dose levels will be evaluated during Part 1 of the study: 300 mg, 900 mg, 1500 mg, and 2000 mg. Q4W administration frequency may be explored during the study.

### Objectives and Endpoints

The primary objective of the study is to evaluate the safety and tolerability and determine the MTD and/or RDE(s) of ANTIBODY A in participants with selected advanced malignancies. The primary objective is evaluated by measuring (1) occurrence of dose-limiting toxicities (DLTs), (2) incidence of treatment-emergent adverse events (TEAEs), assessed by physical examinations, evaluating changes in vital signs, left ventricular ejection fraction (LVEF), and electrocardiograms (ECGs), and clinical laboratory blood and urine sample evaluations, and (3) incidence of TEAEs leading to study drug treatment interruptions and withdrawal of study drug due to adverse events (AEs).

The secondary objectives of the study are: (1) to determine the preliminary efficacy of ANTIBODY A in terms of objective response rate (ORR), disease control rate (DCR), and duration of response (DOR) in participants with selected advanced malignancies; (2) to evaluate the PK of ANTIBODY A in participants with selected advanced malignancies; (3) to evaluate the pharmacodynamics of ANTIBODY A in participants with selected advanced malignancies; (4) to assess the immunogenicity of ANTIBODY A in participants with selected advanced malignancies; and (5) to evaluate the target engagement of ANTIBODY A via receptor occupancy in participants with selected advanced malignancies.

The secondary objectives are evaluated by measuring the following endpoints: (1) Objective response: complete response (CR) or partial response (PR), as determined by the investigator by radiographic disease assessment according to RECIST v1.1; Disease control: CR, PR, or stable disease (SD) as determined by the investigator by radiographic disease assessment according to RECIST v1.1, and DOR: time from earliest date of disease response (CR or PR) until earliest date of disease progression as determined by the investigator by radiographic disease assessment according to RECIST v1.1; (2) PK parameters for ANTIBODY A, including Cₘₐₓ, tₘₐₓ, Cₘᵢₙ, AUC, CL, V_{z}, and t_{1/2}, as deemed appropriate; (3) pharmacodynamics of ANTIBODY A including changes in T-lymphocyte activation and cytokines in the blood as well as intratumoral T-lymphocyte changes; (4) Immunogenicity: defined as the occurrence of ADAs specific to ANTIBODY A; and (5) receptor occupancy in peripheral blood samples.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a disorder in a human subject in need thereof, wherein the disorder is selected from the group consisting of a non-small cell lung cancer (NSCLC), a squamous cell carcinoma of the head and neck (SCCHN), a cervical squamous cell carcinoma and endocervical adenocarcinoma (CESC), an ovarian cancer, a breast cancer, a bladder cancer, a renal cell carcinoma, a melanoma, a gastric adenocarcinoma, an esophageal cancer, a gastroesophageal adenocarcinoma, a malignant pleural mesothelioma, a pancreatic adenocarcinoma, and a colorectal cancer (CRC), wherein the method comprises administering to the human subject a therapeutically effective amount of a bispecific antibody that binds to human programmed death-1 (PD-1) and human transforming growth factor β receptor 2 (TGFβR2), wherein the bispecific antibody comprises:
an anti-human PD-1 binding domain comprising a PD-1 heavy chain variable region and a PD-1 light chain variable region, wherein the PD-1 heavy chain variable region comprises heavy chain CDR1 (HCDR1) comprising the amino acid sequence RFALH (SEQ ID NO:1), heavy chain CDR2 (HCDR2) comprising the amino acid sequence WIDPNTGTPTFAQGVTG (SEQ ID NO:2), and heavy chain CDR3 (HCDR3) comprising the amino acid sequence SLGYCDSDICYPNWIFDN (SEQ ID NO:3), and wherein the PD-1 light chain variable region comprises light chain CDR1 (LCDR1) comprising the amino acid sequence QSISSY (SEQ ID NO:11), light chain CDR2 (LCDR2) comprising the amino acid sequence AAS (SEQ ID NO:12), and light chain CDR3 (LCDR3) comprising the amino acid sequence QQSYSTPPT (SEQ ID NO:13); and
an anti-human TGFβR2 binding domain comprising a TGFβR2 heavy chain variable region and a TGFβR2 light chain variable region, wherein the TGFβR2 heavy chain variable region comprises HCDR1 comprising the amino acid sequence IYAMT (SEQ ID NO:6), HCDR2 comprising the amino acid sequence VISGSGGTTYYADSVKG (SEQ ID NO:7), and HCDR3 comprising the amino acid sequence RGQYRDIVGATDY (SEQ ID NO:8), and wherein the TGFβR2 light chain variable region comprises LCDR1 comprising the amino acid sequence QSISSY (SEQ ID NO:11), LCDR2 comprising the amino acid sequence AAS (SEQ ID NO:12), and LCDR3 comprising the amino acid sequence QQSYSTPPT (SEQ ID NO:13).

2. The method of claim 1, wherein the PD-1 heavy chain variable region comprises the amino acid sequence QVQLVQSGSELKKPGASVKVSCKASGYTFTRFALHWVRQAPGQGLEWMGWIDPNTGT PTFAQGVTGRFVFSLDTSVTTAYLQISSLKAEDTAVYYCARSLGYCDSDICYPNWIFDN WGQGTLVTVSS (SEQ ID NO:4) and the TGFβR2 heavy chain variable region comprises the amino acid sequence EVQLVESGGGLVQPGGSLRLSCAASGFTFDIYAMTWVRQAPGKGLEWVSVISGSGGTT YYADSVKGRFTISRDNSKNTLYLQMNSLRAEDTAVYYCARRGQYRDIVGATDYWGQG TLVTVSS (SEQ ID NO:9).

3. The method of claim 1, wherein the PD-1 light chain variable region and the TGFβR2 light chain variable region each comprise the amino acid sequence DIQMTQSPSSLSASVGDRVTITCRASQSISSYLNWYQQKPGKAPKLLIYAASSLQSGVPSR FSGSGSGTDFTLTISSLQPEDFATYYCQQSYSTPPTFGQGTKVEIK (SEQ ID NO:14).

4. The method of claim 1, wherein the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO:15, the TGFβR2 heavy chain comprises the amino acid sequence set forth in SEQ ID NO:10, and the TGFβR2 light chain light chain comprises the amino acid sequence set forth in SEQ ID NO:15.

5. The method of claim 1, wherein the human subject has a NSCLC.

6. The method of claim 5, wherein the NSCLC has squamous or nonsquamous histology.

7. The method of claim 5, wherein the NSCLC is advanced or metastatic.

8. The method of claim 1, wherein the human subject has a SCCHN.

9. The method of claim 8, wherein the SCCHN comprises a primary squamous tumor of the oral cavity, oropharynx, hypopharynx, or larynx.

10. The method of claim 8, wherein the SCCHN is advanced or metastatic.

11. The method of claim 1, wherein the human subject has a CESC.

12. The method of claim 11, wherein the CESC is advanced or metastatic.

13. The method of claim 1, wherein the human subject has human papilloma virus (HPV)-positive SCCHN or HPV-positive CESC.

14. The method of claim 1, wherein the human subject has an ovarian cancer.

15. The method of claim 14, wherein the ovarian cancer comprises ovarian epithelial carcinoma, fallopian tube carcinoma, primary peritoneal carcinoma, or carcinosarcoma.

16. The method of claim 14, wherein the ovarian cancer is advanced or metastatic.

17. The method of claim 1, wherein the human subject has a breast cancer.

18. The method of claim 17, wherein the breast cancer comprises a triple negative breast cancer.

19. The method of claim 18, wherein the triple negative breast cancer comprises HER2-negative, ER-negative, and PgR-negative breast cancer.

20. The method of claim 1, wherein the breast cancer is advanced or metastatic.

21. The method of claim 1, wherein the human subject has a bladder cancer.

22. The method of claim 21, wherein the bladder cancer comprises urothelial carcinoma.

23. The method of claim 21, wherein the bladder cancer is advanced or metastatic.

24. The method of claim 1, wherein the human subject has a renal cell carcinoma.

25. The method of claim 24, wherein the renal cell carcinoma is advanced or metastatic.

26. The method of claim 1, wherein the human subject has a melanoma.

27. The method of claim 26, wherein the melanoma is cutaneous malignant melanoma.

28. The method of claim 26, wherein the melanoma is advanced or metastatic.

29. The method of claim 1, wherein the human subject has a gastric adenocarcinoma.

30. The method of claim 29, wherein the gastric adenocarcinoma is advanced or metastatic.

31. The method of claim 1, wherein the human subject has an esophageal cancer.

32. The method of claim 31, wherein the esophageal cancer is advanced or metastatic.

33. The method of claim 1, wherein the human subject has a gastroesophageal adenocarcinoma.

34. The method of claim 33, wherein the gastroesophageal adenocarcinoma is advanced or metastatic.

35. The method of claim 1, wherein the human subject has a malignant pleural mesothelioma.

36. The method of claim 35, wherein the malignant pleural mesothelioma is advanced or metastatic.

37. The method of claim 1, wherein the human subject has a pancreatic adenocarcinoma.

38. The method of claim 37, wherein the pancreatic adenocarcinoma is advanced or metastatic.

39. The method of claim 1, wherein the human subject has a CRC.

40. The method of claim 39, wherein the CRC comprises microsatellite stable colorectal cancer (MSS-CRC).

41. The method of claim 39, wherein the CRC comprises deficient mismatch repair (dMMR)/high microsatellite instability (MSI-H) colorectal cancer (dMMR/MSI-H CRC).

42. The method of claim 39, wherein the CRC is advanced or metastatic.

43. The method of claim 1, wherein the human subject has experienced disease progression after prior treatment.

44. The method of claim 43, wherein the prior treatment comprises anti-PD-(L)1 therapy and/or anti-CTLA4 therapy.

45. The method of claim 1, wherein the disorder is nonamenable to curative treatments or procedures.

46. The method of claim 1, wherein the bispecific antibody is administered intravenously.

47. The method of claim 1, wherein the bispecific antibody is administered at a dose of about 100 mg, about 300 mg, about 900 mg, about 1500 mg, or about 2000 mg.

48. The method of claim 1, wherein the bispecific antibody is administered intravenously at a dose of about 100 mg, about 300 mg, about 900 mg, about 1500 mg, or about 2000 mg.

49. The method of claim 1, wherein the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO:15, the TGFβR2 heavy chain comprises the amino acid sequence set forth in SEQ ID NO:10, and the TGFβR2 light chain light chain comprises the amino acid sequence set forth in SEQ ID NO:15, and wherein the bispecific antibody is administered intravenously at a dose of about 100 mg, about 300 mg, about 900 mg, about 1500 mg, or about 2000 mg.

50. The method of claim 1, wherein the bispecific antibody is administered once every two weeks.

51. The method of claim 1, wherein the bispecific antibody is administered intravenously once every two weeks at a dose of about 100 mg, about 300 mg, about 900 mg, about 1500 mg, or about 2000 mg.

52. The method of claim 1, wherein the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO:15, the TGFβR2 heavy chain comprises the amino acid sequence set forth in SEQ ID NO:10, and the TGFβR2 light chain light chain comprises the amino acid sequence set forth in SEQ ID NO:15, and wherein the bispecific antibody is administered intravenously once every two weeks at a dose of about 100 mg, about 300 mg, about 900 mg, about 1500 mg, or about 2000 mg.

53. The method of claim 1, wherein the bispecific antibody is administered once every four weeks.

54. The method of claim 53, wherein the bispecific antibody is administered intravenously once every four weeks at a dose of about 100 mg, about 300 mg, about 900 mg, about 1500 mg, or about 2000 mg.

55. The method of claim 53, wherein the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO:15, the TGFβR2 heavy chain comprises the amino acid sequence set forth in SEQ ID NO:10, and the TGFβR2 light chain light chain comprises the amino acid sequence set forth in SEQ ID NO:15, and wherein the bispecific antibody is administered intravenously once every four weeks at a dose of about 100 mg, about 300 mg, about 900 mg, about 1500 mg, or about 2000 mg.
